(19) United States
(12) Patent Application Publication
(10) Pub. No.: US 2003/0113271 A1
(43) Pub. Date: Jun. 19, 2003

(54) Formulations for pulmonary delivery

(75) Inventors: Derrick Katyama, Denver, CO (US); Mark C. Manning, Denver, CO (US); Kathleen A. Stringer, Denver, CO (US); John E. Repine, Englewood, CO (US)

Correspondence Address: PILLSBURY WINTHROP, LLP, P.O. BOX 10500, MCLEAN, VA, 22102, US

(73) Assignee: University Technology Corporation

(21) Appl. No.: 10/327,476

(22) Filed: Dec. 24, 2002

Related U.S. Application Data
(63) Continuation-in-part of application No. 09/355,522, filed on Oct. 22, 1999, now Pat. No. 6,497,877.
Application No. 09/355,522 is a 371 of international application No. PCT/US98/01948, filed on Jan. 29, 1998.
(60) Provisional application No. 60/036,566, filed on Jan. 29, 1997.

(51) Int. Cl. A61L 9/04; A61K 38/48

(52) U.S. Cl. 424/45; 514/2

(57) ABSTRACT

Formulations for pulmonary delivery that include a protein and a non-physiological surfactant at or above the CMC of the surfactant, and methods for preparing and using the same.

### CROSS-REFERENCE TO RELATED APPLICATIONS

This application claims priority to each of U.S. patent application Ser. No. 09/355,522 filed on Oct. 22, 1999 which is the National Phase of PCT/US98/01948 filed Jan. 29, 1998, and claims priority thereto under 35 U.S.C. 371 and further claims priority to U.S. Patent Application Serial No. 60/036,566 filed Jan. 29, 1997, all of which are incorporated by reference in their entirety herein.

### FIELD OF THE INVENTION

The present invention relates to surfactant formulations for pulmonary drug delivery and methods for using the same. The formulations include a therapeutic protein and a surfactant. More particularly, the present invention relates to formulations that include a plasminogen activator and a surfactant, which formulations can be used to promote fibrinolysis and/or to reduce inflammation.

Table of Abbreviations

| | |
|---|---|
| ARDS | acute respiratory distress syndrome |
| CMC | critical micelle concentration |
| DLS | dynamic light scattering |
| FITC | fluorescein isothiocyanate |
| fMLP | N-formylmethionyl-leucyl-phenylalanine |
| HPF | high powered field |
| IL-1 | interleukin 1 |
| MDI | metered dose inhaler |
| mOD | change in optical density |
| MPO | myeloperoxidase |
| NO | nitric oxide |
| PMA | phorbol ester |
| PPACK | D-Phe-Pro-Arg-chloromethyl ketone HCl |
| ROS | reactive oxygen species |
| SK | streptokinase |
| tPA | tissue plasminogen activator |
| UK | urokinase (uPA) |
| uPA | urokinase plasminogen activator |
| UV | ultraviolet |
| XO | xanthine oxidase |

### DESCRIPTION OF THE RELATED ART

Effective treatment of respiratory diseases and disorders often involves direct delivery of medications to the lungs of the patient. Pulmonary delivery is preferable to oral, intravenous and subcutaneous delivery because it is non-invasive, localized, permits rapid action of medicament, requires a relatively small dosage, is not filtered through the liver of the patient, and produces a low incidence of systemic side effects. However, most medications for the treatment of lung diseases and disorders are not available in formulations suitable for respiratory delivery, in part because lung delivery methods can disrupt the structure of therapeutic proteins.

As one example, acute respiratory distress syndrome (ARDS) contributes significantly to human morbidity and mortality. It is estimated that tens of thousands of patients develop ARDS annually in the United States and more than 50% of them die (Abraham et al., 2000; Artigas et al., 1998). Importantly, ARDS patients who survive hospitalization have no increased risk of subsequent death (Davidson et al., 1999).

Presently there is no effective pharmacotherapy for ARDS. Anti-inflammatory therapies (anti-inflammatory drugs or drugs with the capacity of reducing or modifying inflammatory mediators) such as ketoconazole, lisofylline, and steroids have not been beneficial in patients with ARDS (Siegel, Slutsky), and every inflammatory mediator manipulation trial in ARDS to date has been negative (MacIntyre). Antioxidants such as vitamin E, superoxide dismutase, catalase, N-acetylcysteine, and the xanthine oxidase inhibitor, allopurinol, are also not beneficial to patients with ARDS (Siegel; Slutsky).

Thus, there exists a long-felt need in the art for additional compositions and methods for pulmonary delivery. In particular, there exists a need for preparing formulations for pulmonary drug delivery that preserve the biological activities of therapeutic proteins. To meet this need, the present invention provides formulations suitable for pulmonary delivery, methods for preparing formulations that include a therapeutic protein and a surfactant, and methods for pulmonary delivery of the same.

### SUMMARY OF THE INVENTION

The present invention provides compositions comprising a biologically active protein and a non-physiological surfactant, wherein the non-physiological surfactant is included in an amount greater than the CMC of the surfactant. The compositions are suitable for aerosolization. Optionally, a composition of the invention can further comprise a detectable label.

Also provided are methods for preparing compositions of the invention. In a representative embodiment the method comprises preparing a composition comprising a biologically active protein and a non-physiological surfactant, wherein the non-physiological surfactant is included in an amount greater than the CMC of the surfactant, and wherein the composition is suitable for pulmonary delivery.

A composition of the invention comprises a biologically active protein, such as a therapeutic protein or a diagnostic protein. Preferably, the biologically active protein comprises a human protein. In one embodiment of the invention, the biologically active protein comprises a plasminogen activator, and more preferably a tissue-type plasminogen activator.

Non-physiological surfactants used to prepared compositions of the invention can comprise ionic surfactants or non-ionic surfactants. Preferred surfactants include block copolymer surfactants (e.g., block copolymers of propylene oxide and ethylene oxide, PLURONIC® surfactants, and particularly PLURONIC®-F68 surfactant) and polysorbates (e.g., TWEEN® surfactants, and particularly polysorbate 80 and/or TWEEN®-80 surfactant).

Surfactants used to prepare a composition of the invention are included in an amount greater than the CMC of the surfactant, typically in an amount from 0.01% (w/w) to 0.5% (w/w), an amount from 0.03% (w/w) to 0.5% (w/w), an amount from 0.05% (w/w) to 0.5% (w/w), an amount from 0.1% (w/w) to 0.5% (w/w), or an amount about 0.1% (w/w).

In particular embodiments of the invention, a composition comprises fibrinolytic activity, anti-inflammatory activity, or a combination thereof. Representative anti-inflammatory activities include inhibition of radical oxygen (reactive oxygen species, ROS) production and inhibition of lung leak, as described in the Examples.

When preparing a composition of the invention, aerosolization can be performed using any suitable device. Representative devices include a jet nebulizer, an ultrasonic nebulizer, a metered dose inhaler, and an aerosolization device based on forced passage through a nozzle.

The present invention further provides methods for pulmonary delivery of a biologically active protein to a subject, the method comprising administering an effective amount of a composition, wherein the composition comprises a biologically active protein and a non-physiological surfactant, and wherein the non-physiological surfactant is included in an amount greater than the CMC of the surfactant.

Typically, a composition of the invention is administered to a mammalian subject, preferably a human subject. A subject can display a lung disease or disorder, for example an inflammatory disease or disorder (e.g., acute lung injury, acute respiratory distress syndrome, asthma, bronchitis, or cystic fibrosis), an embolism, or cancer.

In accordance with the disclosed methods, administration of a composition of the invention can be used to treat a lung disease or disorder via pulmonary delivery of the composition to a subject. Thus, the present invention includes pulmonary administration of an effective amount of a composition, whereby lung inflammation is reduced, whereby embolism is reduced, or whereby cancer growth is inhibited. When an aerosol composition of the invention further includes a detectable label, the disclosed methods can further comprise detecting the detectable label.

In one embodiment of the present invention, a method is provided for inhibiting pulmonary inflammation in a subject via pulmonary administration of an effective amount of a composition to a subject, wherein the composition comprises a biologically active tissue-type plasminogen activator and a non-physiological surfactant, wherein the non-physiological surfactant is included in an amount greater than the CMC of the surfactant, and whereby pulmonary inflammation is reduced in the subject.

Accordingly, it is an object of the present invention to provide compositions for pulmonary drug delivery, and methods for preparing and using the same. This object is achieved in whole or in part by the present invention. An object of the invention having been stated above, other objects and advantages of the present invention will become apparent to those skilled in the art after a study of the following description of the invention and non-limiting Examples.

### BRIEF DESCRIPTION OF THE DRAWINGS

FIG. 1 shows the effect of tPA on superoxide anion production by human neutrophils stimulated with PMA in vitro. Adding tPA concentrations of 20-100 μg/ml significantly reduced neutrophil O₂⁻ production compared to values obtained following no additions or addition of 5 μg/ml tPA. Each value is the mean±standard error of three or more determinations. Asterisk, p<0.05.

FIG. 2 shows the effect of L-arginine on O₂⁻ production by human neutrophils in vitro. Adding 175 or 700 μg/ml of L-arginine, a component of the tPA preparation, did not decrease (p>0.05) O₂⁻ production by PMA stimulated neutrophils in vitro. By comparison, adding 1400 or 3500 μg/ml of L-arginine increased O₂⁻ production by PMA stimulated neutrophils in vitro. Each value is the mean±standard error of three or more determinations. Asterisk, p<0.05.

FIG. 3 shows the effect of tPA or PPACK-treated tPA on O₂⁻ production by neutrophils in vitro. Compared to PMA alone, neutrophils treated with tPA or PPACK treated tPA had comparable (p 0.05) decreases in O₂⁻ generation. Neutrophils were pretreated with tPA, PPACK, or PPACK:tPA (5:1 mole ratio) and subsequently activated by PMA. The time course of cytochrome C reduction (O₂⁻ production) was monitored by changes in optical density (mOD). Neither neutrophils alone, tPA alone, PPACK alone, or acetic acid (PPACK vehicle) altered cytochrome C reduction. The Vmax (mOD/minute) or rate of cytochrome C reduction was significantly less following administration of both tPA+PMA, filled square (0.198±0.09), and PPACK:tPA+PMA, open triangle (0.182±0.01), when compared to administration of PMA alone, filled diamond (0.476±0.15); p=0.0004 and 0.006, respectively. In addition, there was no significant difference in Vmax between the tPA+PMA and PPACK:tPA+PMA groups (p>0.05). Data represent the mean of triplicate experiments.

FIG. 4 shows the effect of tPA on produced by xanthine oxidase in vitro. Adding increasing amounts of tPA did not decrease (p>0.05) generation by xanthine oxidase (XO) in vitro. Each value is the mean±standard error of three or more experiments.

FIG. 5 shows the effect of tissue plasminogen activator (tPA) on carrageenan induced edema in rat footpad. Open square, saline alone; filled square, carrageenan alone; filled triangle, 12 mg/kg tPA+carrageenan; filled circle, 6 mg/kg tPA+carrageenan; open circle, 3 mg/kg tPA+carrageenan. Edema index reflects changes in hind paw volume at different times after plantar carrageenan or saline administration. Data represent the mean (±SEM) for ten experiments. Asterisk represents p≦0.05 tPA when compared to carrageenan alone.

FIG. 6 shows the effect of streptokinase (SK) on carrageenan induced edema in rat footpad. Open square, saline alone; filled square, carrageenan alone; filled triangle, 40,000 U/kg SK+carrageenan; filled circle, 20,000 U/kg SK+carrageenan; open circle, 10,000 U/kg SK+carrageenan. Edema index reflects changes in hind paw volume at different times after plantar carrageenan or saline administration. Data represent the mean (±SEM) for ten experiments. Asterisk represents p≦0.05 for SK vs. carrageenan alone.

FIG. 7 shows modulation of IL-1 induced lung leak. Data are presented as the means±SEM. Asterisk represents p<0.05 vs. IL-1 control group. Lung leak induced by L-arginine was not significantly different that that induced by saline alone.

FIG. 8 shows tPA induced inhibition of oxidant production by a rat alveolar macrophage line. Cells were pretreated with tPA (100 μg/ml) or vehicle, and subsequently exposed to phorbol ester (PMA, 1.25 μg/ml), zymosan (ZMA, 60 μg/ml), or opsonized zymosan (opZMA, 60 μg/ml). Large open circle, control; large closed circle, tPA; open square, PMA; closed square, PMA+tPA; open triangle, ZMA; closed triangle, ZMA+tPA; small open circle, opZMA; and small closed circle, opZMA+tPA. Data represent the mean of triplicate estimations.

FIG. 9 shows tPA alone significantly reduced the rate of apoptosis and the percent apoptotic cells at 24 hours. Open circle, cells alone; closed circle, tPA alone; open square, PMA alone; closed square, fMLP alone; open triangle, tPA+PMA; and closed triangle, tPA+fMLP.

FIG. 10 is a bar graph that shows the specific activity of tPA recovered following nebulization performed as described in Example 6. PS-80, TWEEN®-80 surfactant (ICI Americas, Inc. of Bridgewater, N.J.); neb'd, nebulized; F-68, PLURONIC® F68 surfactant (BASF Corporation of Mount Olive, N.J.).

FIG. 11 is a line graph that depicts inhibition of human neutrophil O₂⁻ production by nebulized tPA (100 μg/ml) containing 0.01% TWEEN®-80 surfactant (ICI Americas Inc. of Bridgewater, N.J.). Data are mean (±SEM) of two experiments performed in triplicate. *, p<0.05 when compared to PMA alone.

FIG. 12 is a bar graph that depicts the log of the Aggregation Index for each of the indicated samples. A value <1 (bold line) indicates substantially no aggregation. Nebulized tPA in the absence of surfactant (Genetech of South San Francisco, Calif.) is substantially aggregated when compared to non-nebulized tPA (native). Co-nebulization of tPA and TWEEN®-80 surfactant reduces nebulization-induced tPA aggregation.

FIG. 13 is a bar graph that depicts the percent of recovered tPA having fibrinolytic activity, which was assessed as described in Example 7. Co-nebulization of tPA and at least about 0.1% TWEEN®-80 protects tPA in a biologically active form during the nebulization process.

FIG. 14 is a line graph that depicts the ability of nebulized tPA (neb'd tPA) to inhibit PMA-induced ROS production by neutrophils. The assay was performed as described in Example 7.

### DETAILED DESCRIPTION OF THE INVENTION

I. Definitions

While the following terms are believed to be well understood by one of ordinary skill in the art, the following definitions are set forth to facilitate explanation of the invention.

The terms “composition” and “formulation” are used interchangeably to refer to a product which results by combining or mixing more than one element or ingredient.

The term “aerosolization” refers to a process whereby a liquid formulation is converted to an aerosol. Representative devices for aerosolization include a jet nebulizer, an ultrasonic nebulizer, a metered dose inhaler, and an aerosolization device based on forced passage through a nozzle. The resulting compositions are referred to herein as “aerosol” compositions.

The phrase “suitable for pulmonary delivery” means that a protein included in the composition remains biologically active following pulmonary delivery.

The term “surfactant” refers to an agent having surface active, emulsifying, dispersing, solubilizing, and/or wetting activity.

The term “critical micelle concentration,” abbreviated as “CMC,” refers to a minimal concentration of monomer surfactant at which the surfactant monomers polymerize to form micelles.

The term “micelle” refers to a globular polymer of surfactant monomers.

The term “plasminogen activator” refers to a tissue-type plasminogen activator or to a urokinase plasminogen activator.

The term “tissue-type plasminogen activator,” which is abbreviated as “PA,” refers to a tissue-type plasminogen activator polypeptide, as described herein below, including a tPA pro-peptide (i.e., alteplase or reteplase), and derivatives and structural variants of tPA that contain amino acid substitutions, deletions, additions and/or replacements.

The term “fibrinolytic” refers to an activity that promotes blood clot dissolution involving digestion of insoluble fibrin by plasmin. For example, fibrinolytic activity can comprise activation of plasminogen to plasmin.

The term “anti-inflammatory” refers to an activity that reduces or prevents inflammation.

The term “inflammation” refers to a condition typically characterized by redness, warmth, swelling, and/or pain, which is produced in response to injury or infection. The term “inflammation” encompasses local as well as systemic responses. Local inflammation involves increased blood flow, vasodilation, and/or infiltration leukocytes into tissues, and in some severe cases, intravascular thrombosis, damage to the blood vessels and blood extravasation. Systemic inflammation can involve fever, leukocytosis, and/or release of acute phase reactants into the serum.

The terms “inflammatory disease” and “inflammatory disorder” refer to conditions characterized by inflammation, as well as to symptoms of inflammation resulting from a separate disease or condition. Thus, as described further herein below, the terms “inflammatory disease” and “inflammatory disorder” encompass inflammation associated with acute lung injury, acute respiratory distress syndrome, arthritis, asthma, bronchitis, cystic fibrosis, reperfusion injury artery occlusion, stroke, ultraviolet light induced injury, vasculitis, autoimmune disease, transplantation, and/or leukocyte dysfunction.

The term “about”, as used herein when referring to a measurable value such as an amount, a temporal duration, etc., is meant to encompass variations of ±20% or ±10%, more preferably ±5%, even more preferably ±1%, and still more preferably ±0.1% from the specified value, as such variations are appropriate to perform the disclosed methods.

The terms “a,” “an,” and “the” are used in accordance with convention in the art to refer to one or more.

II. Aerosolization Methods

The present invention provides methods for preparing formulations for pulmonary delivery via nebulization or other means, and the formulations produced thereby. A composition of the invention comprises a protein and a non-physiological surfactant. Surprisingly, the surfactant protects protein structure during aerosolization when included in an amount greater than or equal to the CMC of the surfactant.

Surfactants are believed to protect proteins in solution via one of two common mechanisms. First, they can bind directly to the protein to promote thermodynamic stabilization. This shifts the equilibrium of the native state protein towards the most compact state and away from expanded, aggregation-competent states. When this is the case, maximal stability would be achieved at the stoichiometric ratio of surfactant to detergent, meaning that protection could be optimal well below the critical micelle concentration (cmc). Second, the surfactant could compete with protein molecules for hydrophobic interfaces, such as the air-water interface. Methods for preparing an aerosol create substantial surface area at air-water interface, and optimal protein protection occurs at or just above the cmc.

The disclosure of the present invention reveals the surprising observation that proteins, when included in a formulation including a surfactant at concentrations substantially above the cmc, are protected during aerosolization methods. Also surprisingly, a sufficient amount of surfactant does not include any amount above the cmc, i.e. the cmc is not a threshold concentration. Thus, as described herein below, the present invention further provides methods for determining an amount of surfactant sufficient for protein protection.

A surfactant formulation of the invention can be prepared by combining a protein and one or more surfactants by any suitable technique. For example, a surfactant can be added to a pre-lyophilized protein, to a lyophilized protein, or to a protein that is reconstituted in aqueous or non-aqueous solvent. Proteins and surfactants in the solid phase can be combined using co-grinding techniques, as known in the art. See e.g., Williams et al. (1999) Eur J Pharm Biopharm 48:131-40.

A surfactant used in the compositions and methods disclosed herein comprises a non-physiological surfactant. The term “non-physiological” is used herein to describe a quality of not being found in a mammalian subject. Thus, non-physiological surfactants of the invention exclude surfactant lipids obtained from a mammalian subject, for example SURVANTA® surfactant (Abbott Laboratories Corp. of Abbott Park, Ill.), ALVEOFACT® surfactant (Boehringer Ingelheim of Ingelheim, Germany), and similar physiological surfactants. See e.g., Gunther et al. (2001) Respir Res 2:353-64 and references cited therein. Non-physiological surfactants also exclude recombinantly produced or synthesized surfactants that are normally found in a mammalian subject.

Surfactants used in accordance with the disclosed methods include ionic and non-ionic surfactants. Representative non-ionic surfactants include polysorbates such as TWEEN®-20 and TWEEN-80® surfactants (ICI Americas Inc. of Bridgewater, N.J.); poloxamers (e.g., poloxamer 188); TRITON® surfactants (Sigma of St. Louis, Mo.); sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palnidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; MONAQUAT™ surfactants (Mona Industries Inc. of Paterson, N.J.); polyethyl glycol; polypropyl glycol; block copolymers of ethylene and propylene glycol such as PLURONIC® surfactants (BASF of Mt. Olive, N.J.); oligo (ethylene oxide) alkyl ethers; alkyl (thio) glucosides, alkyl maltosides; and phospholipids. [0055]A composition of the invention comprises an amount of surfactant greater than the CMC and an amount that protects protein structure, as described herein below. For example, the surfactant can be present in a formulation in an amount from about 0.01% to about 0.5% (weight of surfactant relative to total weight of other solid components of the formulation; “w/w”), from about 0.03% to about 0.5% (w/w), from about 0.05% to about 0.5% (w/w), or from about 0.1% to about 0.5% (w/w). For example, in one embodiment of the invention, a formulation comprises tPA protein and TWEEN®)-80 surfactant in an amount about 0.03% (w/w) to about 0.1% (w/w). In another embodiment of the invention, a formulation comprises tPA protein and PLURONIC®-F68 surfactant in an amount about 0.03% (w/w) to about 0.1% (w/w).

A formulation of the invention can also comprise additional agents for protein stabilization, including other surfactants. Thus, a formulation of the invention can comprise a combination of surfactants. A formulation can also comprise sucrose to enhance protein stability and retard aggregation. See e.g., Kim et al. (2001) J Biol Chem 276:1626-33.

The formulations can be aerosolized using any suitable device, including but not limited to a jet nebulizer, an ultrasonic nebulizer, a metered dose inhaler (MDI), and a device for aerosolization of liquids by forced passage through a jet or nozzle (e.g., AERX® drug delivery devices by Aradigm of Hayward, Calif.). For delivery of a formulation to a subject, as described further herein below, an pulmonary delivery device can also include a ventilator, optionally in combination with a mask, mouthpiece, mist inhalation apparatus, and/or a platform that guides users to inhale correctly and automatically deliver the drug at the right time in the breath. Representative aerosolization devices that can be used in accordance with the methods of the present invention include but are not limited to those described in U.S. Pat. Nos. 6,357,671; 6,354,516; 6,241,159; 6,044,841; 6,041,776; 6,016,974; 5,823,179; 5,797,389; 5,660,166; 5,355,872; 5,284,133; and 5,277,175 and U.S. Published Patent Application Nos. 20020020412 and 20020020409.

Using a jet nebulizer, compressed gas from a compressor or hospital air line is passed through a narrow constriction known as a jet. This creates an area of low pressure, and liquid medication from a reservoir is drawn up through a feed tube and fragmented into droplets by the air stream. Only the smallest drops leave the nebulizer directly, while the majority impact on baffles and walls and are returned to the reservoir. Consequently, the time required to perform jet nebulization varies according to the volume of the composition to be nebulized, among other factors, and such time can readily be adjusted by one of skill in the art.

A metered dose inhalator (MDI) can be used to deliver a composition of the invention in a more concentrated form than typically delivered using a nebulizer. For optimal effect, MDI delivery systems require proper administration technique, which includes coordinated actuation of aerosol delivery with inhalation, a slow inhalation of about 0.5-0.75 liters per second, a deep breath approaching inspiratory capacity inhalation, and at least 4 seconds of breath holding. Pulmonary delivery using a MDI is convenient and suitable when the treatment benefits from a relatively short treatment time and low cost.

Optionally, a formulation can be heated to about 25° C. to about 90° C. during nebulization to promote effective droplet formation and subsequent delivery. See e.g., U.S. Pat. No. 5,299,566.

Aerosol compositions of the invention comprise droplets of the composition that are a suitable size for efficient delivery within the lung. Preferably, a surfactant formulation is effectively delivered to lung bronchi, more preferably to bronchioles, still more preferably to alveolar ducts, and still more preferably to alveoli. Thus, aerosol droplets are typically less than about 15 μm in diameter, and preferably less than about 10 μm in diameter, more preferably less than about 5 μm in diameter, and still more preferably less than about 2 μm in diameter. For efficient delivery to alveolar bronchi of a human subject, an aerosol composition preferably comprises droplets having a diameter of about 1 μm to about 5 μm.

Droplet size can be assessed using techniques known in the art, for example cascade, impaction, laser diffraction, and optical patternation. See McLean et al. (2000) Anal Chem 72:4796-804, Fults et al. (1991) J Pharm Pharmacol 43:726-8, and Vecellio None et al. (2001) J Aerosol Med 14:107-14.

A formulation of the invention can further comprise a detectable label or contrast agent (e.g., a radiolabel) so that the biodistribution of the formulation can be determined following pulmonary delivery to a subject. See e.g., Cheng et al. (2001a) J Aerosol Med 14:255-66 and Schermuly et al. (2000) Am J Respir Crit Care Med 161:152-9.

Protein stability following aerosolization can be assessed using known techniques in the art, including size exclusion chromatography; electrophoretic techniques; spectroscopic techniques such as UV spectroscopy and circular dichroism spectroscopy, and protein activity (measured in vitro or in vivo). To perform in vitro assays of protein stability, an aerosol composition can be collected and then distilled or absorbed onto a filter. To perform in vivo assays, or for pulmonary administration of a composition to a subject, a device for aerosolization is adapted for inhalation by the subject.

For example, protein stability can be assessed by determining the level of protein aggregation. Preferably, an aerosol composition of the invention is substantially free of protein aggregates. The presence of soluble aggregates can be determined qualitatively using DLS (DynaPro-801TC, ProteinSolutions Inc. of Charlottesville, Va.) and/or by UV spectrophotometry, as described in Example 6.

In preferred embodiments of the invention, an aerosol composition comprises a fibrinolytic activity, an anti-inflammatory activity, or a combination thereof. Representative methods for assessing fibrinolytic and anti-inflammatory activities are described herein below, and particularly in the Examples.

Fibrinolytic activity of an aerosol composition can be assessed using any suitable technique known in the art. For example, fibrinolytic activity can be assessed in vitro by measurement of the amidolytic activity of plasmin on a chromogenic substrate, as described in the Examples. Fibrinolytic activity can also be assessed in vivo by determining reduction of an embolism. Pulmonary embolism can be monitored by known techniques in the art, including by ventilation/perfusion lung scan, impedance plethysmography, and/or venous compression ultrasound.

Representative techniques for determining an anti-inflammatory activity of an aerosol composition include in vitro assays of reduced neutrophil ROS production and in vivo measurements of reduced lung leak, as described in the Examples.

Protein activity, such as a fibrinolytic or anti-inflammatory activity, of an aerosol composition preferably comprises greater than about 50% or more protein activity, still more preferably greater than about 60% or more, still more preferably greater than about 70% or more, still more preferably greater than about 80% or more, still more preferably greater than about 90% or more, still more preferably greater than about 95% or more, and still more preferably greater than about 99% or more.

Thus, an anti-inflammatory activity of an aerosol composition preferably comprises at least about 50% inhibition of ROS production, for example when measured using an in vitro assay as described in the Examples. More preferably, a surfactant formulation comprises at least about 50% inhibition of ROS production, still more preferably at least about 60% inhibition of ROS production, still more preferably at least about 70% inhibition of ROS production, still more preferably at least about 80% inhibition of ROS production, still more preferably at least about 90% inhibition of ROS production, still more preferably at least about 95% inhibition of ROS production, and still more preferably at least about 99% inhibition of ROS production.

A formulation of the invention preferably comprises at least about 10% respirable dose. The term “respirable dose” refers to the fraction of liquid formulation that is sufficiently aerosolized for pulmonary delivery. Compositions of the invention comprise a respirable dose of at least about 10%, more preferably at least about 20%, still more preferably at least about 30%, still more preferably at least about 40%, still more preferably at least about 50%, still more preferably at least about 60%, still more preferably at least about 70%, still more preferably at least about 80%, still more preferably at least about 90%, and still more preferably at least about 95%.

Pulmonary delivery of an aerosol composition comprising an anti-inflammatory activity to a subject preferably results in about 40% or more suppression of IL-1 induced lung leak, still more preferably greater than about 50% or more, still more preferably greater than about 60% or more, still more preferably greater than about 70% or more, still more preferably greater than about 80% or more, still more preferably greater than about 90% or more, still more preferably greater than about 95% or more, and still more preferably greater than about 99% or more.

III. Proteins for Delivery

A formulation of the invention comprises a therapeutic protein useful for the treatment or prophylaxis of a pulmonary disease or disorder. Representative therapeutic proteins include enzymes and antibodies. A protein used to prepare a formulation suitable for aerosolization can also comprise a detectable label. Optionally, a composition of the invention can comprise a therapeutic protein and a detectable label.

Proteins can be isolated, synthesized, recombinantly produced purified, and characterized using a variety of standard techniques that are known to the skilled artisan. Standard recombinant DNA and molecular cloning techniques used to isolate nucleic acids can be found, for example, in Sambrook et al. (eds.) (1989) Molecular Cloning: A Laboratory Manual. Cold Spring Harbor Laboratory Press, Cold Spring Harbor, N.Y.; Silhavy et al. (1984) Experiments with Gene Fusions. Cold Spring Harbor Laboratory Press, Cold Spring Harbor, N.Y.; Glover & Hames (1995) DNA Cloning: A Practical Approach, 2nd ed. IRL Press at Oxford University Press, Oxford/New York; and Ausubel (ed.) (1995) Short Protocols in Molecular Biology, 3rd ed. Wiley, New York.

III.A. Plasminogen Activators In a preferred embodiment of the invention, a therapeutic protein comprises a plasminogen activator (PA), such as a tissue-type plasminogen activator (tPA) or a urokinase plasminogen activator (uPA).

The plasminogen activators are implicated in fibrin removal. Both cleave the circulating zymogen, plasminogen, to generate the less specific serine protease, plasmin. tPA also has anti-inflammatory activity, as disclosed in U.S. patent application Ser. No. 09,355,522 and No. 60/036,566.

tPA and uPA are homologous proteins that contain similar EGF domains, disulfide-linked structures referred to as Kringles, and a carboxyl terminal Serine Protease (SP) domain. The SP domain is homologous to similar domains in plasma clotting serine proteases, urokinase, and trypsin, and contains the active site for the fibrin-specific serine protease activity.

tPA can be provided as tissue-type plasminogen activator precursor (also called alteplase or reteplase), which is cleaved in vivo to an active two-chain polypeptide. Nucleic acid and protein sequences of representative tPAs and tPA precursors are set forth as GenBank Nos. P00750, NP_{—}127509, NP_{—}00922, and NP_{—}000921. See also Pennica et al. (1983) Nature 301:214-21.

Methods of assaying for various properties of tPA, methods of making derivatives and structural variants of tPA, methods of expressing and purifying tPA, and other information are described in U.S. Pat. Nos. 4,766,075; 4,963,357; 5,094,953; 5,106,741; 5,108,901; 5,149,533; 5,156,969; 5,232,847; 5,242,688; 5,246,850; 5,270,198; 5,275,946; 5,486,471; and 5,556,621. Each of the above-cited references is incorporated herein by reference in its entirety.

uPA can also be provided as a precursor protein or pro-enzyme. Representative uPA nucleic acid and protein sequences are set forth as GenBank Nos. P00749 and CAA26268. See also Riccio et al. (1985) Nucleic Acids Res 13:2759-71 and U.S. Pat. Nos. 4,326,033; 4,370,417; 5,112,755; 5,175,105; 5,219,569; 5,240,845; 5,472,692; 5,519,120; 5,550,213; and 5,571,708, which are incorporated herein by reference in their entirety.

Functional domains of tPA and uPA, including substrate-binding and receptor-binding domains, have been well-characterized. See e.g., Pennica et al. (1983) Nature 301:214-21, Ny et al. (1984) Proc Natl Acad Sci USA 81:5355-9, Gurewich et al. (1988) J Clin Invest 82:1956-62, Appella et al. (1987) J Biol Chem 262:4437-40, Stoppelli et al. (1985) Proc Natl Acad Sci USA 82:4939-43, Magdolen et al. (1996) Eur J Biochem 237:743-51, and Riccio et al. (1985) Nucleic Acids Res 13:2759-71.

Based on the description of plasminogen activator functional domains and methods for assaying the associated functions, a plasminogen activator protein used in accordance with the disclosed methods, derivatives comprising modified PA functions can be readily produced. Derivatives and structural variants of tPA or uPA proteins may contain amino acid substitutions, deletions, additions and/or replacements. For example, such derivatives may contain deletions in the serine protease (SP) domain, and/or mutations that reduce or eliminate the serine protease activity of plasminogen activator. Non-thrombolytic forms of plasminogen activator may be produced by means such as, for example, incubation with a serine protease inhibitor as described in U.S. Pat. No. 5,304,482, formation of a complex with a plasminogen activator inhibitor (PAI), isolation of a plasminogen activator fragment after chemical or enzymatic cleavage, and/or genetic engineering. The proteolytic activity of tPA can be inhibited by PPACK. The tPA-PPACK complex retains an ability to inhibit human neutrophil O₂⁻ production in vitro. See Stringer et al. (1997) Inflammation 21:27-34.

The invention also provides a method of screening structural variants of plasminogen activator for their ability to act as anti-inflammatory agents. Activity as an anti-inflammatory agent may be assayed by oxidant production by an inflammatory cell (e.g., neutrophil, macrophage, monocyte, eosinophil, mast cell, basophil); the carrageenan rat footpad model; and/or interleukin-1 induced pulmonary injury. In addition, structural variants of plasminogen activator may be screened for fibrinolytic activity and/or binding to a receptor for plasminogen activator.

The disclosed formulations comprising a plasminogen activator can also include an inhibitor of a protease released during inflammation by leukocytes (e.g., cathepsin G, chymase, elastase, tryptase. Representative protease inhibitors include but are not limited to α₁-antiprotease, α₁-antitrypsin (AAT), aprotinin, 3,4-dichloro-isocoumarin, diisopropyl fluorophosphate (DFP), α₂-macroglobulin, phenylmethylsulfonyl fluoride (PMSF), plasminogen activator inhibitor (PAI), secretory leukoprotease inhibitor (SLPI), and/or urinary trypsin inhibitor (UTI). See e.g., U.S. Pat. Nos. 5,420,110; 5,541,288; 5,455,229; 5,510,333; and 5,525,623. A composition of the invention can also comprises a plasminogen activator and one or more of an oxidant scavenger (e.g., superoxide dismutase, for example as described in U.S. Pat. No. 4,976,959), a growth factor (for example as described in U.S. Pat. No. 5,057,494) and/or an inhibitor of interleukin-1 (for example as described in U.S. Pat. Nos. 5,075,222; 5,359,032; 5,453,490; 5,455,330; and 5,521,185).

For lung cancer therapies, a therapeutic protein can comprise a tumor suppressor protein, an anti-angiogenic protein, an immunostimulatory protein, antimetabolites, suicide gene products, and combinations thereof. See Kirk & Mule (2000) Hum Gene Ther 11:797-806; Mackensen et al. (1997) Cytokine Growth Factor Rev 8:119-128; Walther & Stein (1999) Mol Biotechnol 13:21-28; and references cited therein.

A protein used to prepare a composition of the invention can also comprise a diagnostic protein. The term “diagnostic protein” refers to a protein whose binding properties are indicative of a particular condition, disease, or disorder. A representative diagnostic protein comprises a protein that specifically binds to, or is indicative of, a lung cancer cell.

IV. Methods for Pulmonary Administration to a Subject

The compositions of the invention can be further formulated according to known methods to prepare pharmaceutical compositions. Suitable formulations for administration to a subject include aqueous and non-aqueous sterile injection solutions which can contain anti-oxidants, buffers, bacteriostats, antibacterial and antifungal agents (e.g., parabens, chlorobutanol, phenol, ascorbic acid, an thimerosal), solutes that render the formulation isotonic with the bodily fluids of the intended recipient (e.g., sugars, salts, and polyalcohols), suspending agents and thickening agents. Surfactant formulations can optionally include a spreading agent such as a fatty alcohol (e.g., cetyl alcohol) or a lung surfactant protein in an amount effective to spread the surfactant formulation on the surface of lung alveoli. Suitable solvents include water, ethanol, polyol (e.g., glycerol, propylene glycol, and liquid polyethylene glycol), and mixtures thereof. The formulations can be presented in unit-dose or multi-dose containers, for example sealed ampoules and vials, and can be stored in a frozen or freeze-dried (lyophilized) condition requiring only the addition of sterile liquid carrier immediately prior to use.

The formulations according to the invention are buffeted to a pH of from about 5 to about 7, preferably about 6. Suitable buffers are those which are physiologically acceptable upon administration by inhalation. Such buffers include citric acid buffers and phosphate buffers, of which phosphate buffers are preferred. Particularly preferred buffers for use in the formulations of the invention are monosodium phosphate dihydrate and dibasic sodium phosphate anhydrous.

The present invention provides that an effective amount of a non-pathogenic virus is administered to a subject. The term “effective amount” is used herein to describe an amount of a non-pathogenic virus sufficient to elicit a desired biological response. For example, when a formulation comprises a plasminogen activator, an effective amount comprises an amount sufficient to promote fibrinolysis, to reduce inflammation, to reduce oxidative injury, and/or to reduce oxidant production. An effective amount can also comprise an amount sufficient to elicit an anti-cancer activity, including cancer cell cytolysis, inhibition of cancer growth, inhibition of cancer metastasis, and/or cancer resistance.

For diagnostic applications, a detectable amount of a composition of the invention is administered to a subject. A “detectable amount,” as used herein to refer to a diagnostic composition, refers to a dose of such a composition that the presence of the composition can be determined in vivo following pulmonary administration.

Actual dosage levels of active ingredients in a composition of the invention can be varied so as to administer an amount of the composition that is effective to achieve the desired diagnostic or therapeutic outcome for a particular subject. Administration regimens can also be varied. A single injection or multiple injections can be used. The selected dosage level and regimen will depend upon a variety of factors including the activity of the therapeutic composition, formulation, the route of administration, combination with other drugs or treatments, the disease or disorder to be detected and/or treated, and the physical condition and prior medical history of the subject being treated. Determination and adjustment of an effective amount or dose, as well as evaluation of when and how to make such adjustments, are known to those of ordinary skill in the art of medicine.

For additional guidance regarding formulation, dose and administration regimen, see Berkow et al. (1997) The Merck Manual of Medical Information, Home ed. Merck Research Laboratories, Whitehouse Station, N.J.; Goodman et al. (1996) Goodman & Gilman's the Pharmacological Basis of Therapeutics, 9th ed. McGraw-Hill Health Professions Division, New York; Ebadi (1998) CRC Desk Reference of Clinical Pharmacology. CRC Press, Boca Raton, Fla.; Katzung (2001) Basic & Clinical Pharmacology, 8th ed. Lange Medical Books/McGraw-Hill Medical Pub. Division, New York; Remington et al. (1975) Remington's Pharmaceutical Sciences, 15th ed. Mack Pub. Co., Easton, Pa.; Speight et al. (1997) AverV's Drug Treatment: A Guide to the Properties, Choice, Therapeutic Use and Economic Value of Drugs in Disease Management, 4th ed. Adis International, Auckland/Philadelphia, Pa.

Pulmonary administration of a surfactant composition of the present invention can be combined with other techniques for pulmonary delivery, for example carbon dioxide enhancement of inhalation therapy (see e.g., U.S. Pat. No. 6,440,393) and bronchodilation (see e.g., U.S. Pat. No. 5,674,860 and U.S. Published Patent Application No. 20020151597). A treatment regimen can also comprise pulmonary delivery with other delivery routes (e.g., oral and intravascular delivery).

V. Applications

The surfactant compositions of the present invention, and methods for pulmonary administration of the compositions to a subject, are useful for the treatment of a disease or disorder of the lung, such as an infection, an immunodeficiency syndrome, an inflammatory disease, an autoimmune disease, a neoplasm, or cancer. In particular, the present invention provides improved methods for formulating therapeutic proteins for pulmonary delivery.

It is envisioned that the disclosed methods are generally useful in mammalian subjects, including human and non-human subjects. The term “subject” generally refers to mammalian animals, including livestock animals (e.g., ungulates, such as bovines, buffalo, equines, ovines, porcines and caprines), primates (e.g., monkeys, chimpanzees, baboons, and gorillas), as well as rodents (e.g., mice, hamsters, rats and guinea pigs), canines, felines, and rabbits. The term “non-human” is meant to include all mammalian animals, especially mammals and including primates other than human primates.

In a one embodiment of the invention, surfactant formulations comprising plasminogen activator are prepared. Preferably, such compositions have fibrinolytic activity, anti-inflammatory activity, or a combination thereof. Plasminogen activator compositions of the invention can also have anti-cancer activity and/or can be used to ameliorate unwanted side-effects of anti-cancer therapies.

As disclosed herein, plasminogen activator can inhibit leukocyte generation of oxygen radicals (e.g., hydroxides, peroxides, superoxides) by a mechanism that is independent of thrombolytic activity and scavenging of oxygen free radicals. By separating the thrombolytic and the anti-inflammatory functions of plasminogen activator, the present invention provides a method of reducing tissue damage due to oxidative injury (e.g., reperfusion injury) while mitigating complications from excessive bleeding, such as stroke and intracerebral hemorrhage. Moreover, because the present invention does not inhibit neutrophil migration and infiltration, use of plasminogen activator as an anti-inflammatory agent does not interfere with processes mediated at least in part by neutrophils such as, for example, wound healing or tissue remodeling, which is a shortcoming of existing steroidal and non-steroidal anti-inflammatory agents.

Representative therapeutic embodiments of the methods of the present invention are described herein below, including methods for pulmonary administration to modulate fibrinolytic balance, to reduce inflammation, and to inhibit cancer growth. Representative embodiments of the invention for diagnosis and/or imaging or pulmonary diseases and disorders. The present invention also provides that the disclosed therapeutic and diagnostic methods can be used in combination. In addition, the disclosed methods can be used in combination with therapeutic and diagnostic methods known in the art. For example, for the treatment of ARDS, a aerosol composition of the invention can be used in combination with other ARDS treatments, including tPA administration via an alternate administrative route (e.g., parenteral administration, such as intravascular injection).

V.A. Fibrinolytic Balance

Plasminogen activators play an important physiological role in the regulation of thrombolysis. This action is exploited therapeutically in conditions such as, for example, acute myocardial infarction, pulmonary embolism, and thrombotic stroke.

An equilibrium between two opposing reactions, coagulation and fibrinolysis, maintains an intact vascular endothelium. To stop blood loss from a leaking blood vessel, blood clots form a hemostatic plug at the site of a break in the vessel wall. But if the blood clot obstructs flow through a blood vessel, myocardial infarction, pulmonary embolism, or thrombotic stroke can result.

The interruption of flow through the blood vessel will lead to tissue ischemia. In this condition, the tissue is deprived of oxygen and becomes jeopardized, a state in which the tissue is injured but still potentially viable. If the hypoxic condition is maintained for a period of several hours, the tissue becomes necrotic and cannot recover. Therefore, it is important that reperfusion, the restoration of blood flow, be accomplished as soon as possible to minimize tissue necrosis. See Hansen (1995) Circulation 91:1872-85. Plasminogen activator can be used to induce thrombolysis in patients with acute myocardial infarction. See e.g., Dakik & Nasrallah (2001) Heart Dis 3:362-4, Kehl et al. (1996) Intensive Care Med 22:968-71, and Munkvad (1993) Dan Med Bull 40:383-408. This benefit is due to blood clot fibrinolysis and timely opening of the infarct-related artery. Thrombolysis of brachial arteries has also been reported (Grieg, 1998).

Surfactant formulations of plasminogen activator of the present invention can be administered to promote fibrinolysis of pulmonary embolisms. As disclosed herein, aerosol compositions retain fibrinolytic activity and are effectively administered to the lung.

V.B. Inflammation

Reperfusion is also associated with harmful effects of neutrophil activation and tissue infiltration. The nature of the neutrophil-mediated injury is not fully characterized but is in part due to the production of superoxide anion (O₂⁻) and/or related oxidative products. This sequence of events (activation of white blood cells, release of toxic mediators, and resultant pathophysiology in the host) is common to many inflammatory diseases.

The present invention also provides compositions and methods for treating conditions associated with oxidative injury. For example, aerosol compositions comprising tissue plasminogen activator can be used to reduce cell and/or tissue damage due to oxidative injury, and to inhibit oxidant production by leukocytes. Tissue at risk of oxidative injury may include blood-perfused tissue and inflamed tissue.

Inflammatory diseases and disorders that can be treated using the disclosed compositions and methods include but are not limited to acute lung injury, acute respiratory distress syndrome, arthritis, asthma, bronchitis, cystic fibrosis, reperfusion injury artery occlusion, stroke, ultraviolet light induced injury, and/or vasculitis. The inflammation can be symptomatic of a separate disease or condition, such as autoimmune disease and transplantation. Inflammatory diseases and disorders also include those conditions characterized by leukocyte dysfunction. The inflammation can be acute, chronic, or temporary inflammation. See e.g., Weissmann et al. (1982) Ann N Y Acad Sci 389:11-24, Goldstein et al. (1982) Ann N Y Acad Sci 389:368-79, Janoff (1985) Annu Rev Med 36:207-16, Hart & Fritzler (1989) J Rheumatol 16:1184-91, Doring (1994) Am J Respir Crit Care Med 150:S114-7, Demling (1995) Annu Rev Med 46:193-202, Hansen (1995) Circulation 91:1872-85, Dakik & Nasrallah (2001) Heart Dis 3:362-4, Kehl et al. (1996) Intensive Care Med 22:968-71, and Munkvad (1993) Dan Med Bull 40:383-408.

In one embodiment of the invention, nebulized tPA formulations are used to treat acute respiratory distress syndrome (ARDS). ARDS is an acute inflammatory disease that involves the sequestration of neutrophils in the lungs (Cooper et al., 1988; Fulkerson et al., 1996). Neutrophils are the primary instigators of lung injury via the generation of ROS (Idell et al., 1989; Idell et al., 1991). Fibrin deposition is also a hallmark of ARDS and may contribute to neutrophil retention in the lung (Cooper et al., 1988; Fulkerson et al., 1996; Idell et al., 1991). This phenomenon may be due to impairment of the intrinsic ability of lung epithelial cells to produce plasminogen activator with an associated increase in plasminogen activator inhibitor (PAI)-1, which antagonizes tPA (Idell et al., 1989; Idell et al., 1991).

As disclosed in the Examples, uPA lacks the inhibitory effect on neutrophil ROS production that tPA possesses. Thus, targeted pulmonary delivery of tPA for the treatment of ARDS may be particularly advantageous by providing fibrinolytic and anti-inflammatory activities to the lung while minimizing systemic fibrinolysis.

In another embodiment of the invention, a surfactant formulation for the treatment of ARDS can include both uPA and tPA. uPA is the predominant plasminogen activator in the lungs and is depressed in ARDS (Bertozzi et al., 1990; Marshall et al., 1990). Thus, treatment can include pulmonary delivery of both tPA and uPA.

Reduced tissue inflammation can be assayed by detecting proteins induced by inflammation, such as cytokines, monokines, receptors, and proteases. For example, histamine can be measured using a fluorescent assay described by Shore et al. (1959) J Pharmacol Exp Ther 127:182-186. Nitric oxide can be measured using a chemiluminescent assay described by Hybertson (1994) Anal Lett 127:3081-3093.

Reduced inflammation can also be assessed by measuring a reduction in oxidant production, including oxidant production by neutrophils, macrophages, monocytes eosinophils, mast cells and/or basophils. Representative methods for assaying the production of oxidants by inflammatory cells are described in the examples. Neutrophil function can also be assayed using techniques known in the art, for example, as described by Bell et al. (1990) Br Heart J 63:82-7, Riesenberg et al. (1995) Br Heart J 73:14-9, Zivkovic et al. (1995) J Pharmacol Exp Ther 272:300-9.

V.C. Lung Cancer

Reduced levels of tissue-type plasminogen activator are also observed in the core and periphery of non-small cell lung carcinoma (Pavey et al., 1999), suggesting that administration of tPA could be ameliorative in this clinical context as well. Representative methods for detecting and monitoring the progress of non-small cell lung carcinoma are described in U.S. Pat. Nos. 6,242,204; 5,795,871; and 5,756,512; among other places. [000x] Reduced levels of plasminogen activity are observed in patients undergoing conventional cancer therapies, for example, in response to chemotherapy (Ruiz et al., 1989) and radiation treatment (Ts'ao et al., 1983), suggesting that administration of nebulized PA can be used to minimize thromboembolic side-effects associated with these therapies.

V.D. Pulmonary Imaging

An aerosol composition of the invention can also comprise a detectable label. Preferably, the detectable label can be detected in vivo, for example by using any one of techniques including but not limited to magnetic resonance imaging, scintigraphic imaging, ultrasound, or fluorescence. Thus, representative detectable labels include fluorophores, epitopes, radioactive labels, and contrast agents.

In one embodiment of the invention, the detectable label is a protein, e.g., a fluorescent protein. Alternatively, the detectable label is conjugated to a protein to be administered. For example, a composition of the invention can comprise a diagnostic protein which is conjugated or otherwise bound to a detectable label. Representative detectable labels, labeling methods, and imaging systems suitable for pulmonary imaging and diagnosis are described in Desai (2002) Clin Radiol 57:8-17, McLoud (2002) Clin Chest Med 23:123-36, and McWilliams et al. (2002) Oncogene 21:6949-59, among other places.

### EXAMPLES

The following Examples have been included to illustrate modes of the invention. Certain aspects of the following Examples are described in terms of techniques and procedures found or contemplated by the present co-inventors to work well in the practice of the invention. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the invention.

### Example 1

Plasminogen Activator Inhibits Oxidant Production

Summary. The following example shows that tissue plasminogen activator inhibits super oxide production by human neutrophils. See Stringer et al. (1997) Inflammation 21:27-34. tPA significantly reduced O₂⁻ production by PMA stimulated human neutrophils in vitro. The inhibitory effect of tPA was not dependent on tPA proteolytic activity, not related to L-arginine in its formulation, and not a consequence of its direct scavenging of O₂⁻. These observations show that tPA has another action, inhibition of neutrophil O₂⁻ production, which may be used to reduce neutrophil O₂⁻ production and prevent oxidative injury.

These results indicate that tPA acts directly on the neutrophil to reduce O₂⁻ production, independent of fibrinolytic activity. These observations could have important clinical implications for optimizing the efficacy of tPA in the management of myocardial infarction as well as other inflammatory processes where a contribution by neutrophil derived O₂⁻ is likely. Indeed, the possibility that tPA might have anti-inflammatory effects is supported by our related in vivo findings shown below.

Recovery and Purification of Human Neutrophils. Human neutrophils were isolated from the whole blood of a single, healthy, drug-free donor using a percoll density gradient (POLYMORHPREP™ from Nycomed Pharma of Oslo, Norway) (Ferrante and Thong, 1980). Cells were then suspended in Krebs-Ringers-Phosphate-Dextrose (KRPD) buffer (serum-free), counted, and assessed for viability using trypan blue exclusion. Tissue plasminogen activator (tPA, alteplase from Genentech of South San Francisco, Calif.) was reconstituted following the manufacturer's instructions using sterile water for injection to produce a final concentration of I mg/ml. All experiments were performed at 37° C. and pH 7.4, under sterile conditions.

Measurement of Neutrophil O₂⁻ Generation by tPA. tPA was added to the neutrophil suspension in sufficient quantities to produce final concentrations of 5, 20, 40, or 100 μg/ml. The effect of L-arginine on neutrophil O₂⁻ generation was also evaluated because L-arginine is a precursor of nitric oxide (NO) and the standard formulation of tPA contains 700 mg L-arginine/20 mg tPA. L-arginine (Sigma Chemical Co. of St. Louis, Mo.) concentrations of 175, 700, 1400, or 3500 μg/ml were evaluated that corresponded to the tPA concentrations used above. Release of O₂⁻ by neutrophils (5×10⁶ cells/ml) stimulated with phorbol myristate acetate (PMA, 1.25 μg/ml) was determined during a 30 minute incubation in the absence or presence of each concentration of tPA or L-arginine. O₂⁻ generation was determined spectrophotometrically by measuring superoxide dismutase (SOD) inhibitable horse heart ferricytochrome C reduction (Babior et al., 1973; Fantone and Kinnes, 1983). Experiments were performed in triplicate.

PPACK Inhibition of tPA. D-Phe-Pro-Arg-chloromethyl ketone HCl (PPACK, available from Calbiochem of San Diego, Calif.) is an irreversible serine protease inhibitor, that inhibits the proteolytic activity of tPA in vitro (Lijnen et al., 1984). tPA was incubated in the presence of PPACK at varying molar ratios (PPACK:tPA: 5:1, 25:1, 100:1, or 1000:1) for 10 minutes, after which PPACK:tPA complexes or tPA alone (100 μg/ml) were incubated with plasminogen (375 μg/ml) for 5 hours in a cell incubator (5% CO₂ in air) at 37° C. Subsequently, 50 μl of each of the incubated samples were subjected to 7.5% acrylamide gel electrophoresis along with tPA (100 μg/ml), plasminogen (375 μg/ml), and plasmin (1 U/ml). Each gel was run at 30V for 16 hours and protein bands were visualized by Coomasie blue stain.

The effect of the PPACK:tPA complex on human neutrophil O₂⁻ production was also examined. Briefly, the cell suspension (5×10⁶ cells/ml) was divided into four groups: tPA (100 μg/ml); PPACK:tPA (5:1); PPACK (140 μM) alone, and PPACK vehicle (10 mM acetic acid). Cells (250 μl of 5×10⁶/ml) from each group were then plated into a 96-well microtiter plate and incubated for 30 minutes at 37° C. in a cell incubator. Cells were then exposed to PMA (1.25 μg/ml) so that the following conditions were met (in triplicate): tPA alone, tPA+PMA, PPACK:tPA alone, PPACK:tPA+PMA, PPACK alone, PPACK+PMA, PMA alone, PPACK vehicle, and cells alone. The plate was then incubated for an additional 30 minutes at 37° C. in a cell incubator after which it was placed in a SPECTRAMAX® plate reader (Molecular Devices of Menlo Park, Calif.) and O₂⁻-production was measured as cytochrome C reduction (550 nm OD) every five minutes for 2 hours (Waud et al., 1975). The kinetic disposition of each treatment was compared.

Measurement O₂⁻ of Scavenging by tPA. The ability of tPA to scavenge O₂⁻ was determined by measuring reduction of cytochrome C during a 30 minutes incubation with purified xanthine oxidase (1.6 U/ml) and hypoxanthine in the presence or absence of tPA (concentrations previously mentioned) (Waud et al., 1975). Experiments were performed in triplicate.

Data Analysis. The mean and standard error of the mean (±SEM) for data were determined for each experiment. Treatment groups were compared to each other and to positive and negative controls by analyses of variance and unpaired student's t tests. Concentration dependent effects were assessed by linear regression followed by an F test for significance. A p value of less than 0.05 was considered significant.

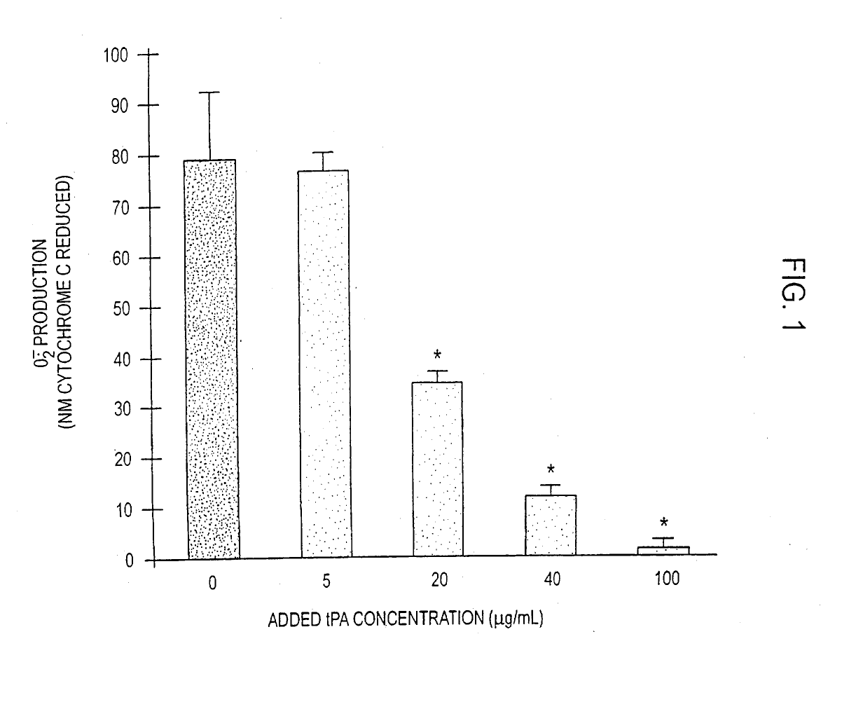

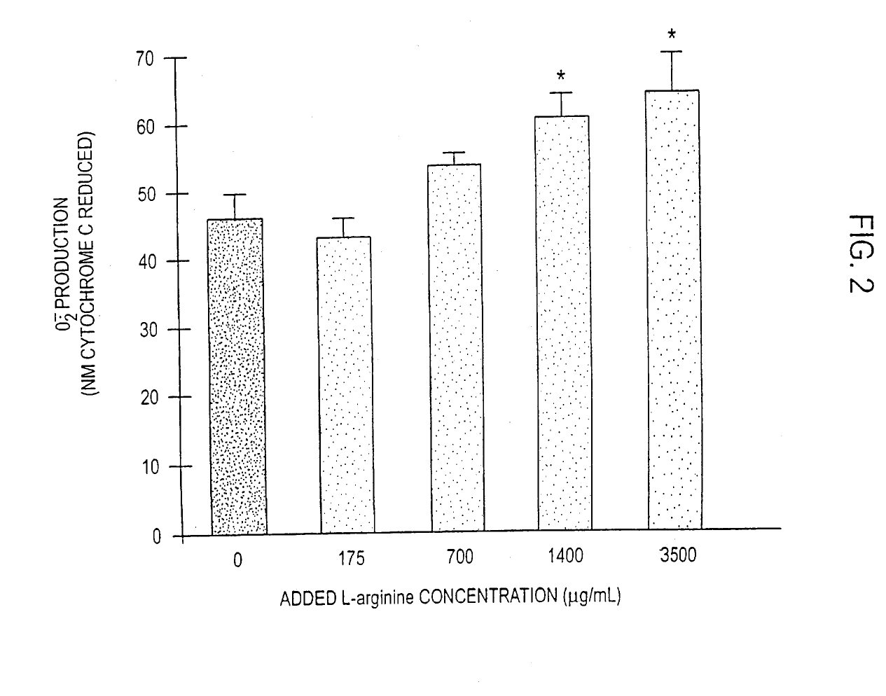

Effect of tPA on Neutrophil O₂⁻ Generation In vitro. Adding increasing amounts of tPA significantly (p 0.025) and progressively decreased O₂⁻ production by human neutrophils stimulated by PMA in vitro (FIG. 1). In contrast, adding L-arginine, a component of the tPA formulation, did not decrease (p>0.05) O₂⁻ production by neutrophils stimulated with PMA (FIG. 2). Neither tPA nor L-arginine altered neutrophil O₂⁻-production by unstimulated neutrophils.

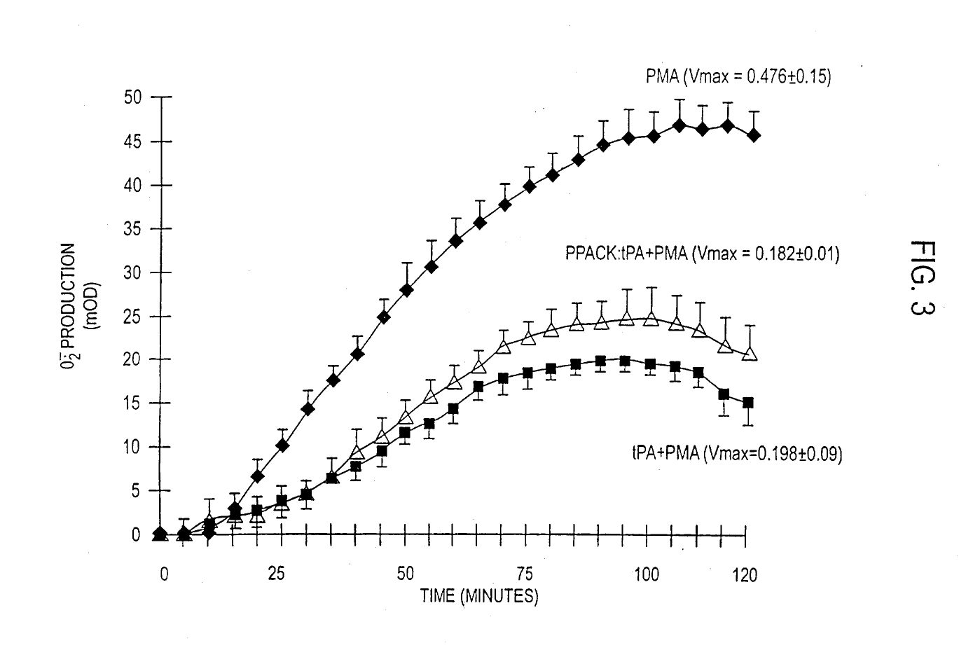

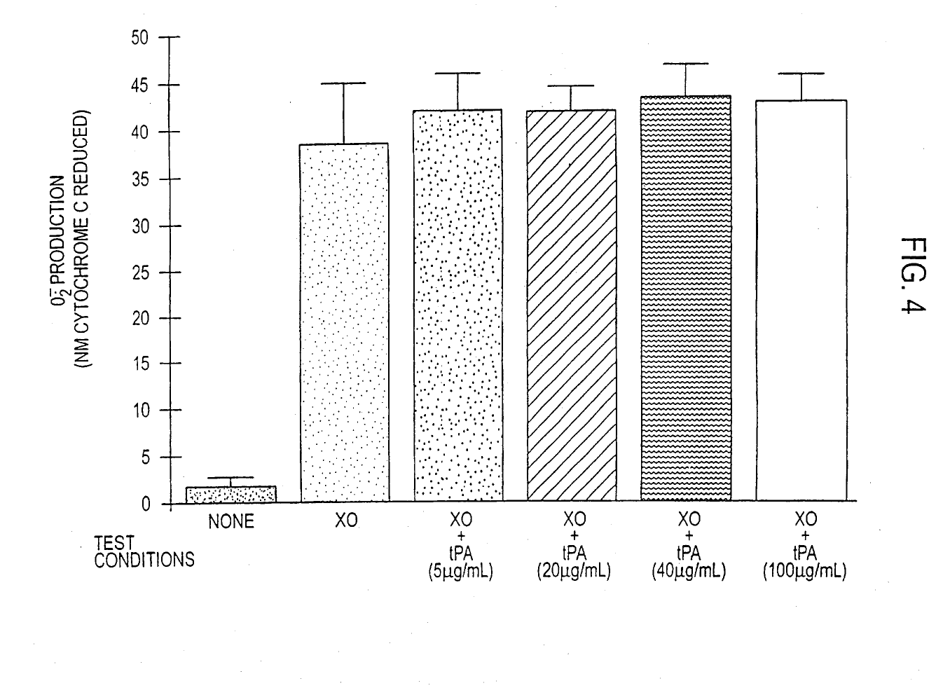

Effect of tPA Proteolytic Activity on O₂⁻ Production. tPA promoted conversion of plasminogen to plasmin. tPA mediated conversion of plasminogen to plasmin was inhibited by PPACK in a concentration dependent fashion. Based on these results, the mole:mole (PPACK:tPA) ratio used in the subsequent experiments was 5:1. In these studies, both tPA and PPACK-treated, proteolytically inactivated, tPA comparably inhibited O₂⁻ production by neutrophils stimulated with PMA (FIG. 4). In addition, analysis of the kinetics Of O₂⁻ production showed that both tPA and proteolytically inactivated tPA decreased the Vmax of O₂⁻ production (i.e. rate of cytochrome reduction) similarly (FIG. 3).

Effect of tPA on O₂⁻ Generation by Xanthine Oxidase In vitro. Adding tPA did not decrease O₂⁻ concentrations produced by xanthine oxidase (XO) in vitro (FIG. 4).

### Example 2

Tissue Type Plasminogen Activator Reduces Inflammation in the Carrageenan—Induced Rat Footpad Model

Summary. This example shows that tPA, but not streptokinase (SK), can reduce inflammation in an in vivo model, the carrageenan-induced rat footpad model. See Stringer et al. (1997a) Free Radic Biol Med 22:985-8. Carrageenan, a mucopolysaccharide derived from Irish sea moss, is a phlogistic agent that provokes a local antigenic inflammatory response which is primarily attributed to neutrophil mediated injury and is highly reproducible (Vinegar et al., 1969; Vinegar et al., 1976; Vinegar et al., 1987). This model has been used extensively to evaluate the anti-inflammatory effects of such drugs as the non-steroidal anti-inflammatory drugs, corticosteriods, and more recently superoxide dismutase (Ando et al., 1991; Vinegar et al., 1987; Winter and Flataker, 1965).

Mechanisms by which tPA could influence carrageenan-induced footpad inflammation and edema include inhibition of neutrophil infiltration into the footpad, inflammatory mediator release, including neutrophil-generated O₂⁻, and/or vascular permeability. The first possibility is unlikely since there was no difference between the plasminogen activators in regard to the magnitude of neutrophil infiltration into the footpad. Generation of O₂⁻ exerts important pro-inflammatory effects, including deesterification of phospholipids resulting in increased vascular permeability like that observed in ischemia-reperfusion injury (Deby and Goutier, 1990). While not wishing to be bound by any particular mode of operation, the anti-inflammatory activity of tPA likely involves its capacity to reduce O₂⁻ production by neutrophils.

In contrast to tPA, SK enhanced inflammation as reflected in the increase in edema index at the later time points and had no effect on neutrophil O₂⁻ production. Plasminogen activators are known to bind to endothelial cell surfaces (Hajjar et al., 1987), and thus the pro-inflammatory effect of SK may involve a direct effect on blood vessels. Consistent with this role, myocardial infarction patients treated with SK experience some degree of hypotension (a occurrence that is not observed in patients treated with tPA). The vasodilatory action of SK may contribute to the enhancement of edema.

Animals. The right hind foot volume of male Sprague-Dawley rats weighing between 200-250 grams was determined using water-displacement prior to carrageenan or carrageenan vehicle (saline) injection. Following initial baseline (pretreatment) foot volume determinations, the rats. were lightly anesthetized using methoxyflurane (Pittman-Moore of Mundelein, Ill.) and 0.10 ml of 1.5% (w/v) carrageenan (Sigma Chemical Co. of St. Louis, Mo.) in sterile normal saline, or saline (0.10 ml, sterile normal saline) was injected into the plantar tissue of the right hind paw. Volume of the injected paw was measured at 30 minutes, and then every hour for 6 hours thereafter.

Treatments. Both SK and tPA were reconstituted according to manufacturers' instructions. Baseline footpad volume measurements were made immediately prior to carrageenan or saline administration.

Tissue plasminogen activator (tPA, also called alteplase, obtained from Genentech of South San Francisco, Calif.) was prepared in each of three doses (3, 6, and 12 mg/kg body weight). Half of each dose was given intraperitoneally (i.p.) 10 minutes prior to footpad carrageenan injection. The second half of the dose was administered 2.5 hours after the first half of the tPA dose. This treatment regimen was considered necessary to account for the short half-life of tPA, which is approximately 5 minutes (Tebbe et al., 1989).

L-arginine (Sigma Chemical Co. of St. Louis, Mo.) was included in tPA formulations (from Genentech of South San Francisco, Calif.) to enhance solubility. The effect of L-arginine, which is a precursor of nitric oxide (NO), was also assessed for anti-inflammatory activity. Doses of L-arginine (0.11, 0.22, 0.44 g/kg body weight, i.p.) utilized correspond to those contained in the tPA doses.

Streptokinase (SK, KABIKINASE® streptokinase from Kabi-Vitrum, Sweden) was prepared as each of three single doses (10,000, 20,000, or 40,000 U/kg body weight, i.p.) and was administered 10 minutes prior to the carrageenan footpad injection.

Histological Examination. Upon completion of the experiments, the animals were sacrificed and their paw removed, fixed in formalin, sectioned, and stained with hematoxylin and eosin. Sections were examined and assessed for neutrophil infiltration by an individual unaware of the sample identities. Neutrophils from both treatment and control groups were visualized at 40× magnification. The number of neutrophils in representative fields of view (also called high powered fields) were counted.

Data Analysis. Calculation of the edema index: An edema index was calculated for each footpad as a measure of inflammation. This was determined by subtracting the weight of the water-filled tube following insertion of the paw at each time point from the weight of the water-filled tube. Edema induces a greater displacement of water. The time zero (pretreatment) foot volume was then subtracted from each time point so that changes in volume reflected those associated with edema. The mean (±SEM) edema index for each time point for each group was determined. The edema indexes for each PA or L-arginine group were compared to carrageenan control group at each time point using a Mann-Whitney two sample test. In all cases, a p value less than 0.05 was considered significant.

Histological examination. The mean (±SEM) neutrophil count per high powered field (HPF) was determined for each treatment and compared to the carrageenan control using analysis of variance (ANOVA).

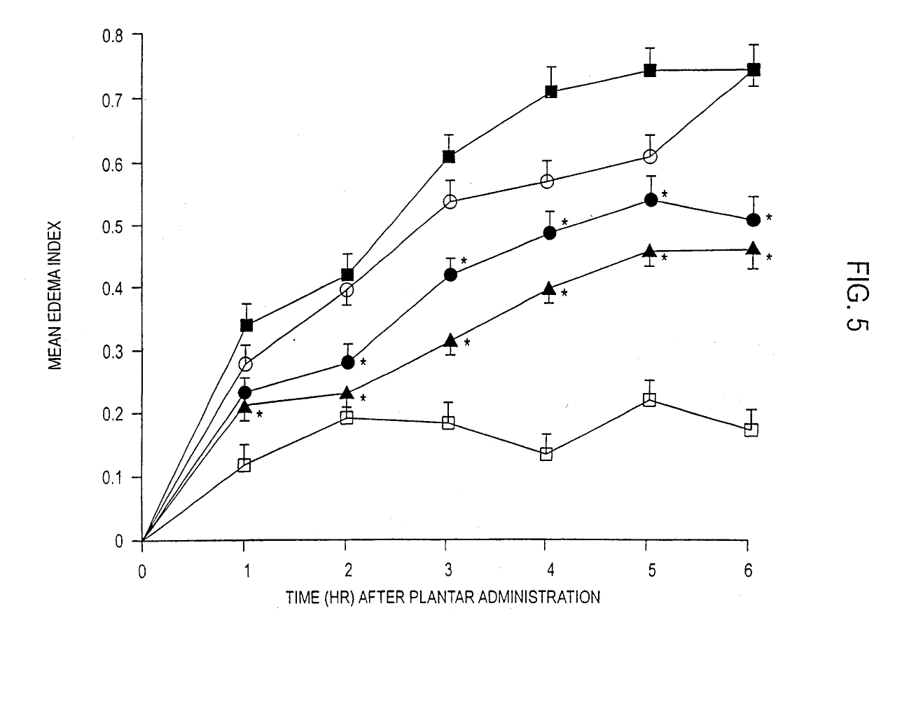

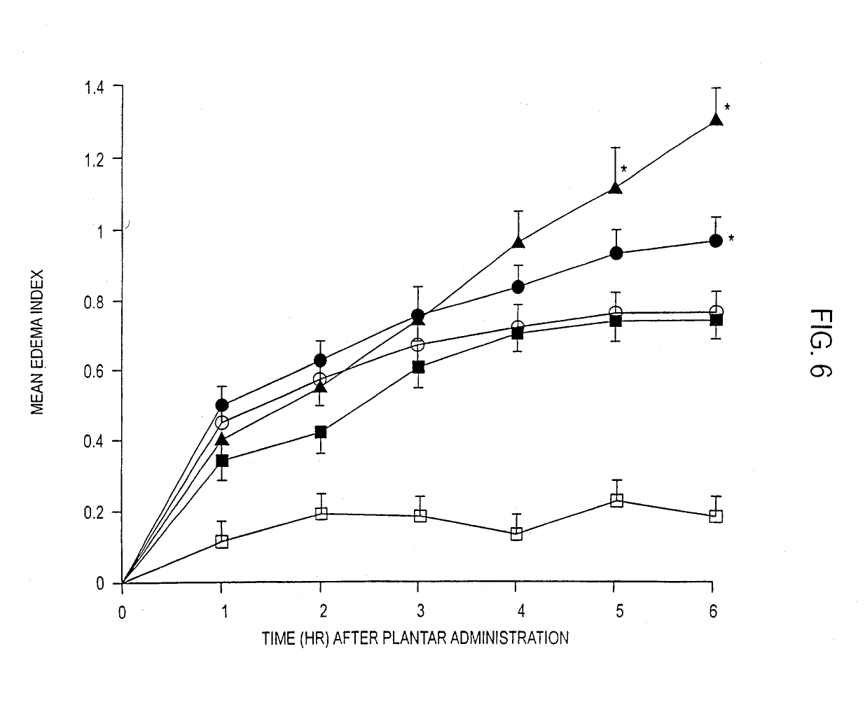

tPA Reduces Inflammation. Carrageenan-induced edema when injected into the rat footpad (FIGS. 5-6). tPA reduced edema in a dose-dependent manner (FIG. 5). At a dose of 12 mg/kg body weight, tPA reduced edema at all time points (p<0.05) while 6 mg/body weight reduced edema beginning at the two hour time point (p<0.05); an effect that occurred prior to the second dose of tPA. The two highest doses of SK, 20,000 and 40,000 U/kg body weight, enhanced edema at the latter time points (≧5 hours) (FIG. 6). By contrast, L-arginine, one of the constituents of the tPA formulation, had no significant effect on edema at any time.

Histological examination of the footpads revealed no significant differences in the number of neutrophils (mean±SEM) between the treatment groups and carrageenan control (carrageenan control: 30.7±0.65 cells/HPF; tPA: 35.0±12.6 cells/HPF; SK: 41.2±16.9 cells/HPF). Notably, the vehicle control footpads had no neutrophil infiltration.

L-arginine did not affect edema, indicating that L-arginine, which is an excipient in the tPA formulation, does not contribute to the anti-inflammatory effect of tPA. Consistent with this observation, L-arginine also does not alter neutrophil O₂⁻ production in vitro.

### Example 3

Tissue Type Plasminogen Activator Reduces Inflammation in the IL-1 Induced Pulmonary Injury Model

Summary. This example shows that tPA can reduce inflammation in the IL-1 induced pulmonary injury model. Intraperitoneal administration of tPA increases lung tissue tPA levels and decreases acute lung injury. Consistent with the effects of tPA in the carrageenan-induced rat footpad model (Example 2), tPA did not abrogate neutrophil infiltration induced by an inflammatory stimulus in vivo. The inhibition of lung injury may be due to an inhibitory effect of tPA on neutrophil O₂⁻ production.

Treatment Regimens. Tissue plasminogen activator (tPA, also called alteplase, available from Genentech of South San Francisco, Calif.) was reconstituted according to the manufacturer's instructions. The total dose was 12 mg/kg body weight given intraperitoneally (i.p.); 6 mg/kg was administered 10 minutes before IL-1 and 6 mg/kg was given 2.5 hours later. This regimen was chosen based on the short half-life of tPA (Tebbe et al., 1989) and based on the dose response study of Example 2. In addition, this dose of tPA does not increase the activated partial thromboplastin time (aPTT) in rats. See Example 2 and Korninger & Collen (1981) Thromb Haemost 46:561-5. To control for possible effects of L-arginine contained in the formulation used, a corresponding dose of L-arginine (440 mg/kg body weight, i.p.) (Sigma Chemical Co. of St. Louis, Mo.) was administered similarly.

Determination of tPA Concentration in the Lung. To determine the effect of systemic administration of tPA on lung tPA concentrations, six male Sprague-Dawley rats (300-400 gm) were given tPA as described in Example 2 and then, five hours later, the lower left lobe of the lung was removed following euthanasia with methoxyflurane. Samples were stored at −80° C. until assay. Subsequently, samples were thawed and homogenized with ice-cold homogenization buffer (20 mM HEPES/glycerol buffer, pH 7.5), containing protease inhibitors (2 mM EDTA, 2 mM EGTA, 5 μg/ml aprotinin, 10 μM leupeptin, 1 mM PMSF) and centrifuged at 15,000×g for 45 minutes. The protein concentration of each supernatant was determined essentially as described in Lowry et al. (1951) J Biol Chem 193:265-275. Aliquots containing 100 μg protein were subjected to 7.5% polyacrylamide gel electrophoresis and transferred to nitrocellulose essentially as described by Towbin et al. (1979) Proc Natl Acad Sci USA 76:4350-4. These membranes were blocked with 3% skim milk in TNS buffer (15 mM Tris, pH 7.4, 150 mM NaCl, 0.1% Tween-20) overnight and then incubated with an antibody specific for tissue plasminogen activator (1:50 dilution of an anti-tPA sheep polyclonal antibody, affinity purified IgG) (Enzyme Research of South Bend, Ind.) for 60 minutes at 25° C. Blots were then rinsed five times for 5 minutes each with wash buffer (3% skim milk in TNS) and incubated with a secondary polyclonal antibody (1:10,000 dilution of rabbit anti-sheep horseradish peroxidase) (Jackson ImmunoResearch of West Grove, Pa.) for 30 minutes at 25° C. Following five rinses (5 minutes each) with wash buffer, immunoblots were visualized by application of enhanced chemiluminescence (ECL) Western blotting reagents (Pierce of Rockford, Ill.) and exposure to autoradiographic film. Immunolabeled tPA was identified by comparison to a known concentration of tPA (1 μM) run on the same gel.

Interleukin-1 Induced Acute Lung Injury. Ten minutes before intratracheal instillation of IL-1 (50 ng/0.5 ml of rhIL-1α, available from R&D Systems of Minneapolis, Minn.) or vehicle (0.5 ml sterile saline), tPA or L-arginine was administered to male (300-400 gm) Sprague-Dawley rats essentially as described by Leff et al. (1993) Am J Physiol 265:L501-6 and Leff et al. (1994) Am J Physiol 266:L2-8. L-arginine was assessed because it is a precursor of nitric oxide synthesis in vivo and it is contained in the tPA formulation that was used.

After tPA or L-arginine administration, and anesthesia with methoxyflurane (Pitman-Moore of Mundelein, Ill.), a 1-cm neck incision was made and the trachea was exposed by blunt dissection. A 25-gauge angiocatheter was inserted through the tracheal wall and the Teflon catheter advanced without the needle into the trachea. Saline (0.5 ml) or IL-1 (50 ng) in saline (0.5 ml) was administered followed by two 3-ml puffs of air to ensure good distal delivery of the cytokine. See Koh et al. (1995) J Appl Physiol 79:472-8, Leff et al. (1993) Am J Physiol 265:L501-6, and Leff et al. (1994) Am J Physiol 266:L2-8. Soft tissue was re-opposed and the neck incision sutured with three interrupted 3-0 silk sutures. Five hours after IL-1α administration, lung leak, lung myeloperoxidase (MPO) activity, and lung lavage neutrophil counts were determined essentially as described by Krawisz et al. (1984) Gastroenterology 87:1344-50, Leff et al. (1993) Am J Physiol 265:L501-6, and Leff et al. (1994) Am J Physiol 266:L2-8.

Determination of Lung Leak and MPO Activity. Four and one-half hours after intratracheal instillation of saline or IL-1, rats were anesthetized by intraperitoneal administration of a mixture of ketamine (90 mg/kg body weight) and xylazine (5 mg/kg body weight) and ¹²⁵I-BSA (1.0 μCi in 0.5 ml) was administered intravenously. Twenty-five minutes thereafter, rats were ventilated using a small animal respirator (Harvard Apparatus, Inc. of Holliston, Mass.) during laparotomy, thoracotomy, and right ventricular injection of heparin (200U in 0.2 ml). Right ventricular blood samples were obtained, lungs were perfused blood free with PBS and excised. Radioactivity in right lungs and blood samples were measured using a gamma counter. Lung leak index was estimated as counts per minute (cpm) of ¹²⁵I in the lung divided by cpm in 1.0 ml of blood. Left lungs were assayed for MPO activity using o-dianiside as substrate. Six rats were utilized in the saline group (control), ten rats in the IL-1 group, and six rats in the tPA and IL-1 group.

Determination of Lung Lavage Neutrophils. Five hours after tPA administration and instillation of saline or IL-1 as described, rats were anesthetized by intraperitoneal administration of ketamine (90 mg/kg body weight) and xylazine (7 mg/kg body weight). The trachea in each animal was cannulated with an indwelling 16 gauge stub adaptor tube, and then saline (two×3.0 ml) was injected slowly and withdrawn (to lavage lungs). Recovered lavage fluid was centrifuged (250×g for 5 minutes) and the cell pellet was resuspended in 1.0 ml of lavage supernatant. Red blood cells were lysed using hypotonic saline. The total number of leukocytes were counted using a COULTER® counter (Coulter Electronics, Inc. of St. Hialeah, Fla.), a CYTOSPIN® apparatus (Shandon Southern Instruments Limited of Cheshire, England) was used to prepare the cells, and the samples were stained with Wright-Giemsa to determine the percentage and total number of neutrophils. Ten rats were utilized in the IL-1 alone and tPA+IL-1 experiments, while six rats were in the saline group (control).

Data Analysis. Data were analyzed using a one-way analysis of variance with a Student-Newman-Keuls test of multiple comparisons. A p value of less than 0.05 was accepted as being statistically significant.

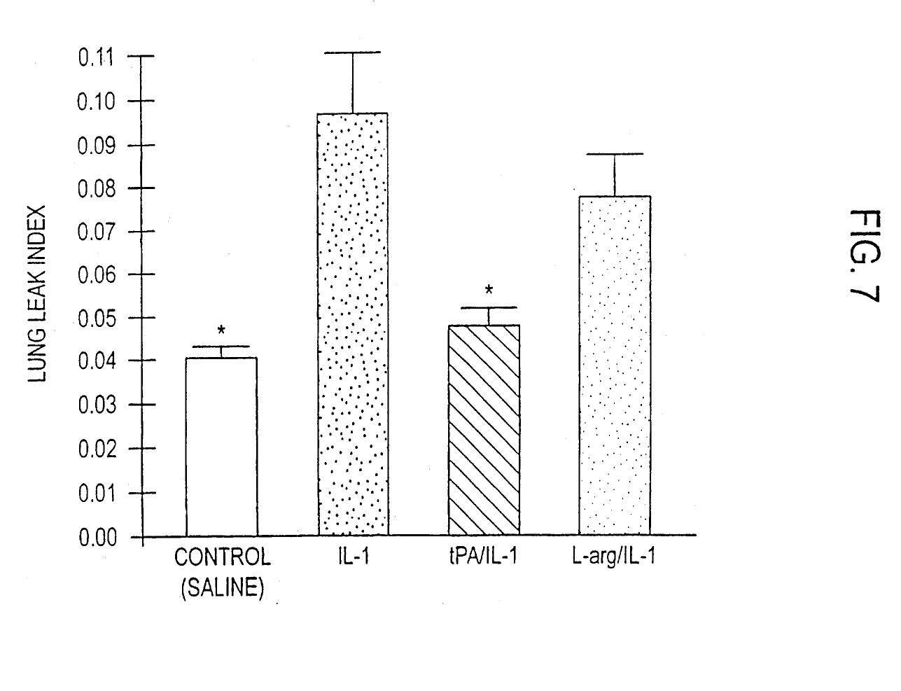

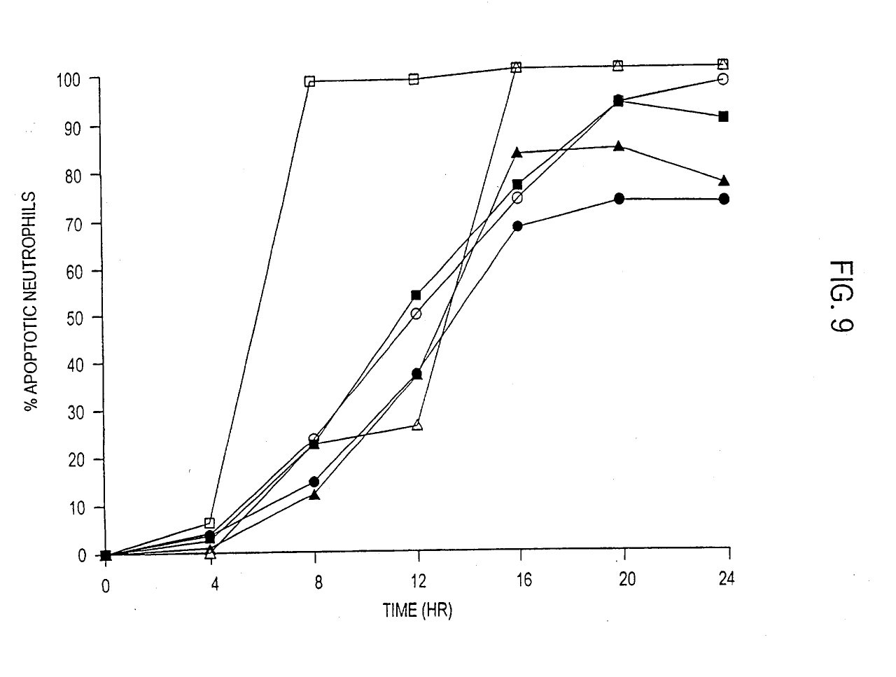

Rats treated with tPA (12 mg/kg body weight, i.p.) had increased lung tPA levels (measured at 5 hours) compared to untreated rats. Rats treated with tPA (12 mg/kg body weight, i.p.) showed an approximately 80% reduction in lung leak compared to untreated rats given IL-1 intratracheally (FIG. 7). Lung leak in rats given L-arginine (440 mg/kg body weight, i.p.) along with IL-1 was not different from lung leak in rats given IL-1 (FIG. 9). In contrast, rats given both tPA and IL-1 had the same number of lavage neutrophils and lung MPO activities as untreated rats given IL-1 intratracheally (Table 1).

TABLE 1
Effect of tPA on Lung Lavage Neutrophils
(PMNs) and Lung MPO Activity

| | Lung lavage | Lung lavage | MPO in whole |
|---|---|---|---|
| | PMNs | PMNs | lung |
| Treatment | (% total cells) | (total #, millions) | (U/gm left lung) |
| control* | 3 ± 1 | 0.003 ± 0.001 | 0.6 ± 0.2 |
| IL-1 | 95 ± 1⁺ | 2.9 ± 0.4⁺ | 11.2 ± 2.9⁺ |
| tPA + IL-1 | 95 ± 1⁺\hat{ } | 2.7 ± 0.4⁺\hat{ } | 11.1 ± 1.6⁺\hat{ } |

*mean ± SEM of six determinations.
⁺value significantly different (p < 0.05) from control value; mean ± SEM of ten determinations.
\hat{ }value not significantly different (p > 0.05) from value obtained for rats given IL-1 alone; mean ± SEM of six determinations.

Lung leak, lung myeloperoxidase (MPO) activity, and lung lavage neutrophil counts were increased in the IL-1 group when compared to the control group (saline). Intraperitoneal administration of tPA (12 mg/kg body weight) increased lung tPA concentration and reduced acute lung leak in rats given IL-1 intratracheally (p<0.01). Lung leak index for sham treatment was 0.040±0.001 (n=6), IL-1 treatment was 0.10±0.01 (n=10), and tPA+IL-1 treatment was 0.050±0.002 (n=6). In contrast, tPA administration did not change the IL-1 induced increases in lavage neutrophils (sham treatment was 3±1×10³ cells, IL-1 treatment was 2.9±0.4×10⁶ cells, and tPA+IL-1 treatment was 2.7±0.4×10⁶ cells) or lung MPO activity (sham treatment was 0.6±0.2 U/gm lung, IL-1 treatment was 11.2±2.9 U/gm lung, and tPA+IL-1 was 11.1±1.6 U/gm lung).

### Example 4

tPA Reduces Activator-Induced Oxidant Production in Macrophages

Chemiluminescence was used to measure the oxidative burst of rat alveolar macrophages (NR 8383 cells). Oxidant production was determined by luminol chemiluminescence, which was measured using a luminometer (LUMISTAR™, available from BMG Lab Technologies Inc. of Durham, N.C.) essentially as described by Archer et al. (1989) J Appl Physiol 67:1912-21. Experiments were conducted in an opaque 96-well plate at 37° C. Suspensions of macrophages (100 μl of 5 million cells/ml) were plated in the presence or absence of tPA (100 μg/ml) 60 minutes prior to exposure to an activator (PMA, zymosan, or opsonized zymosan). Prior to the addition of activator, 200 μl of buffered luminol solution (0.1 μLM) containing horseradish peroxidase (0.5 mg/ml) was added to each well and chemiluminescent light emission was determined (baseline was measured at time 0). Following addition of the activator, chemiluminescent light emission was measured every 10 minutes for two hours. The experiments were performed in triplicate. The assay has a detection limit of approximately 100 nM hydrogen peroxide.

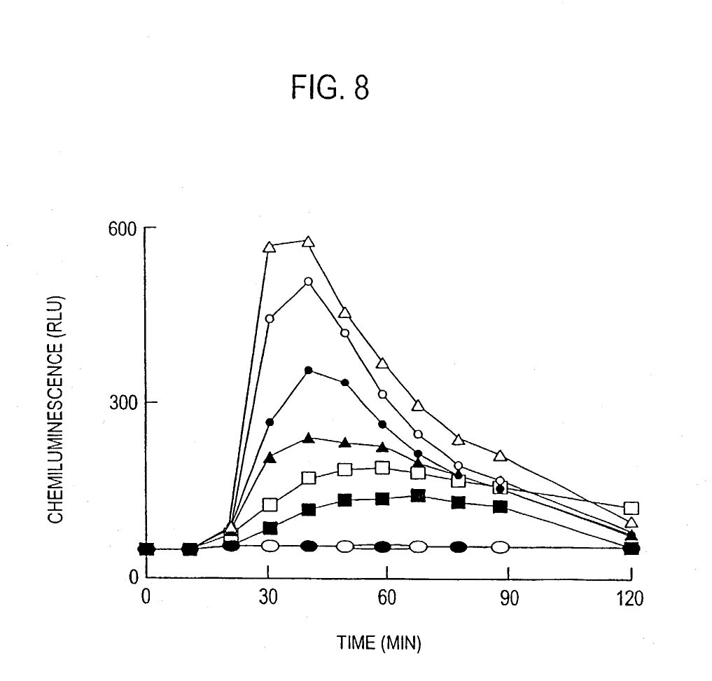

Addition of an activator resulted in an increase in oxidant production by the macrophages (FIG. 8). tPA reduced activator induced oxidant production, demonstrating that the ability of tPA to inhibit oxidant production is not selective for neutrophils but, instead extends to different types of leukocytes.

### Example 5

tPA Reduces Neutrophil Apoptosis

Neutrophils were isolated from the whole blood of a single, healthy, medication-free individual using venipuncture and methods previously described by Stringer et al. (1997b) Inflammation 21:27-34. Cells (1×10⁶ cells/ml) were suspended in Krebs-Ringers-Phosphate-Dextrose (KRPD) buffer and equally divided between two tubes. To one tube, tissue plasminogen activator (tPA) was added to a final concentration of 100 μg/ml. The cell suspension (200 μl) was placed in each well of a 96-well microtiter plate and the plate was incubated (37° C., 5% CO₂) for 30 minutes. Following the incubation, phorbol myristate acetate (PMA, 1.25 μg/ml) or formyl-methionyl-leucyl-phenylalanine (fMLP, 5 μM) was added to wells so that the following conditions were met: cells alone, tPA alone, tPA+PMA, tPA+fMLP, PMA alone, or fMLP alone. The plate was then incubated again (37° C., 5% CO₂) for 30 minutes.

The percent apoptotic cells was determined at time 0 (immediately following incubation), and at 4, 8, 12, 16, 20, and 24 hours thereafter. At each time point, cells (25 μl) were removed from each well of the microtiter plate and placed into a glass tube with 1 μl of ethidium bromide/acridine orange (4 μg/ml each). Cells (10 μl) were then placed on a microscope slide with a cover slip. Cells were viewed under a microscope (100×) equipped with a fluoroscein filter. For each assessment, cells (n=100) were counted and “scored” as either “live apoptotic,” “live normal,” “dead apoptotic,” or “dead normal,” as described by Duke (1995) in Current Protocols in Immunology, ed. Coligan, John Wiley & Sons, New York, pp. 3.17.1-3.17.33.

tPA alone significantly reduced the rate of apoptosis and the percent apoptotic cells at 24 hours (FIG. 9). The rate and magnitude of apoptosis was significantly enhanced by PMA, while fMLP had no effect. The addition of tPA significantly slowed the rate and reduced the magnitude of apoptosis in PMA-treated cells, and reduced the rate and magnitude of apoptosis in fMLP-treated cells.

### Example 6

Formulation of tPA For Pulmonary Delivery

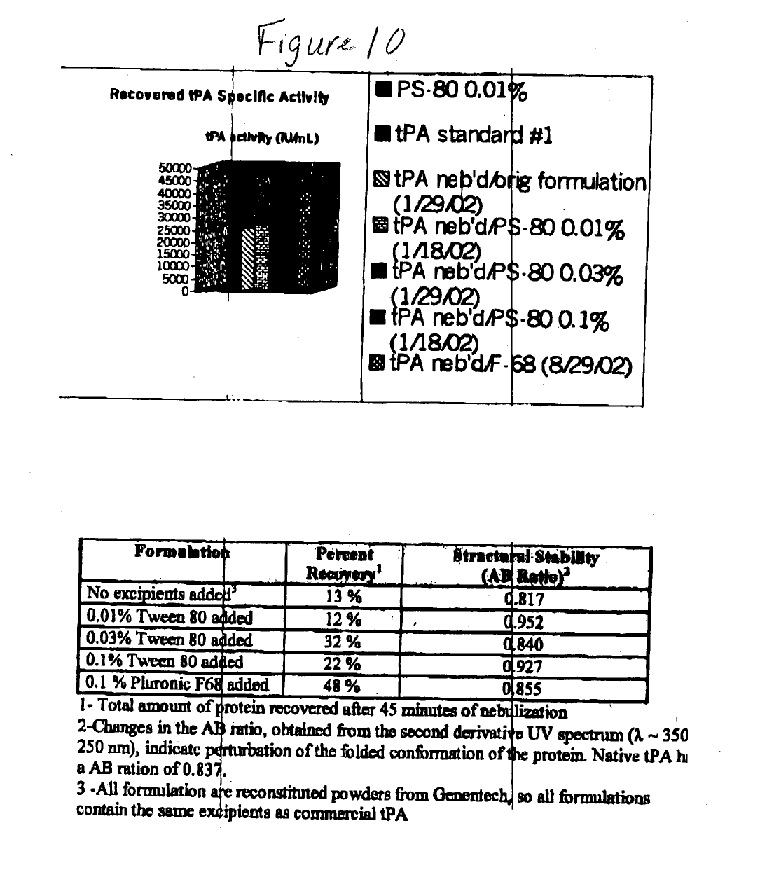

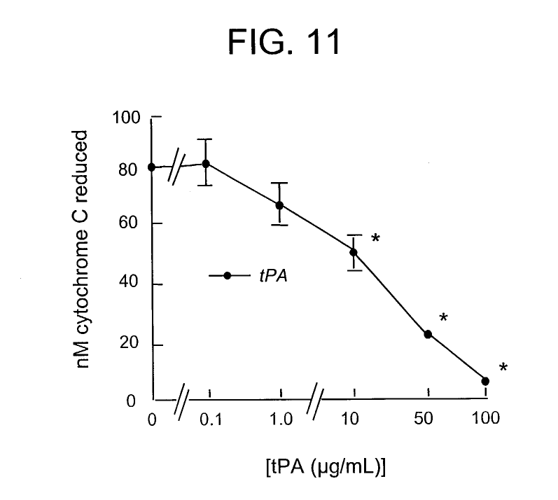

The surfactant TWEEN®-80 surfactant (ICI Americas Inc. of Bridgewater, N.J.) was added to tPA formulations. This markedly increased the stability of tPA during nebulization resulting in >97% recovery of protein. This formulation also retained its ability to inhibit PMA-induced neutrophil ROS production (FIG. 11). Additionally, FIG. 10 is a bar graph that shows the specific activity of tPA recovered following nebulization performed as described in this example. Characterization for soluble aggregates by dynamic light scattering (DLS) was hampered by the presence of micelles. These results indicate that small amounts of TWEEN®-80 surfactant protected tPA during nebulization. Unexpectedly, the surfactant was protective when used at concentrations above the CMC.

TWEEN®-80 surfactant is a commonly used surfactant in the pharmaceutical industry with an extremely good safety profile. To determine an amount of TWEEN®-80 surfactant sufficient to maintain tPA stability during nebulization, tPA (ACTIVASE® tPA, available from Genentech of South San Francisco, Calif.) was reconstituted with sterile water at a concentration of 1 mg/ml according to the manufacturer's instructions. TWEEN®-80 surfactant was added to 5-ml aliquots of tPA to produce final concentrations of 0.01%, 0.03%, and 0.1% TWEEN®-80 surfactant. tPA formulations containing PLURONIC®) surfactant (BASF of Mt. Olive, N.J.) were similarly prepared. The protein concentration of each sample was determined by UV spectrophotometry (DU-64 spectrophotometer, available from Beckman Instruments of Fullerton, Calif.) essentially as described in Dunn et al. (1994) Methods Mol Biol 36:225-43.

Each sample was nebulized using a jet nebulizer (Side Stream nebulizer with Pulmo Aide compressor, available from DeVilbiss of Somerset, Pa.) until the reservoir was empty. As controls, native tPA, the original tPA formulation (without surfactant, obtained from Genetech of South San Francisco, Calif.), and formulation vehicles were also nebulized and the mist collected and assayed. The mist of each sample was collected in a 50-ml conical tube and again assayed for protein concentration by UV spectrophotometry. The mean (±SEM) protein concentration and the corresponding coefficient of variation (CV) for each formulation and control was determined.

Percent protein recovery was determined by dividing the protein concentration following nebulization by the initial protein concentration and multiplying by 100 (Table 2). Each formulation and control sample was evaluated on ten separate occasions to ensure reproducibility. Structural integrity of nebulized tPA was determined by calculating the AB ratio. Preservation of protein structure is observed as a minimal change in the AB ratio following nebulization when compared with the AB ratio of the same formulation prior to nebulization. In contrast, a deviation of the AB ratio following nebulization reflects protein disruption. As shown in Table 2, improved tPA recovery, while substantially maintaining tPA structural stability, was observed following nebulization with at least about 0.03% TWEEN®-80 surfactant and with PLURONIC® F69 surfactant.

Recovery and Structural Stability of Nebulized tPA

| | Percent | Structural Stability |
|---|---|---|
| Formulation | Recovery¹ | (AB Ratio)² |
| No excipients added³ | 13% | 0.817 |
| 0.01% TWEEN ®-80 surfactant added | 12% | 0.952 |
| 0.03% TWEEN ®-80 surfactant added | 32% | 0.840 |
| 0.1% TWEEN ®-80 surfactant added | 22% | 0.927 |
| 0.1% PLURONIC ® F69 surfactant | 48% | 0.855 |
| added | | |

¹Total amount of protein recovered after 15 minutes of nebulization
²Changes in the AB ratio, obtained from the second derivative UV spectrum (□˜350-250 nm), indicate perturbation of the folded conformation of the protein. Native tPA has a AB ratio of 0.837.
³All formulation are reconstituted powders that are commercially available from Genentech (South San Francisco, California) and thus include the same excipients

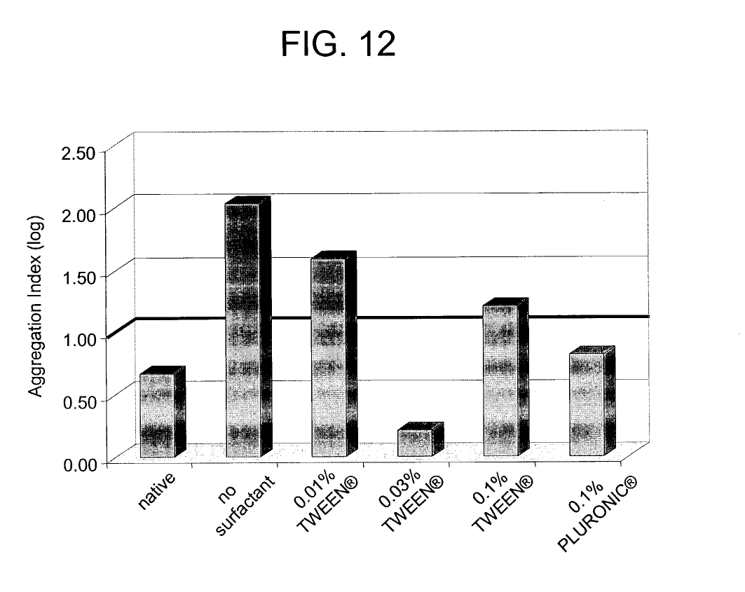

The presence of soluble aggregates was qualitatively assessed using dynamic light scattering (DLS) (DynaPro-801TC, ProteinSolutions, Inc. of Charlottesville, Va.). The presence of soluble aggregates was also assayed by UV spectrophotometry. The Aggregation Index, which is determined from the absorbance at 280 nm and 350 nm, is a gross measure of the extent of aggregation of a protein solution. Each of the formulations containing either TWEEN®-80 surfactant or PLURONIC® surfactant showed reduced aggregation in solution (FIG. 12).

The CMC of TWEEN®-80 surfactant (ICI Americas Inc. of Bridgewater, N.J.) is 0.007%. The lowest of TWEEN®-80 surfactant (ICI Americas Inc. of Bridgewater, N.J.) concentration used (0.01%), which is slightly above the CMC, confers only partial protection of tPA, as evidenced by the formation of particulate matter.

The tPA/surfactant formulation can be frozen for storage. tPA activity, including fibrinolytic and anti-inflammatory activity, is maintained following storage at −30° C. See Wiernikowski et al. (2000) Lancet 355:2221-2.

### Example 7

Fibrinolytic Activity of Nebulized tPA

Nebulized tPA was prepared as described in Example 6. Fibrinolytic activity of nebulized tPA was determined using a CHROMOGENIX® assay (Chromogenix AB Corporation of Molndal, Sweden) adapted for a microtiter plate reader. The principle of the assay is based on the activation of plasminogen to plasmin by tPA. This reaction is markedly increased in the presence of fibrin (tPA stimulator). The fibrinolytic activity of tPA was determined by measuring the amidolytic activity of plasmin on the chromogenic substrate, S-2251 (H-D-Val-Leu-Lys-pNA.2HCl). The release of p-nitroaniline (pNA) was determined at the dual wavelengths, 405 nM and 490 nM, using a microtiter plate reader.

The correlation between the change in absorbance and the activity of tPA was linear within 0.25-10 lU/ml. tPA standards were made by diluting stock tPA (50 lU/ml) in Tris buffer (Tris 0.5M, pH=8.3) to produce final concentrations of 0-10 lU/ml. To each of three wells of a 96-well microtiter plate, sample (100 μl of nebulized reformulated tPA, nebulized original tPA or nebulized vehicle), plasminogen (0.375 μg/ml)/S-2251 (5 mM)/Tris buffer (100 μl) and tPA stimulator (fibrinogen 0.6 mg/ml, 100 μl) were added. A blank was also be plated in triplicate, which included all of the previous components with the exception of tPA stimulator. Standards (0, 0.5, 1.0, 2.5, 5.0, 7.5, and 10 lU/ml; 10 μl of each) were also plated in triplicate. The plate was read on a microtiter plate reader (ThermoMax, available from Molecular Devices Corp. of Sunnyvale, Calif.). tPA activity for each sample was determined from the standard curve plot (activity vs. absorbance), which was calculated automatically by the plate reader's software.

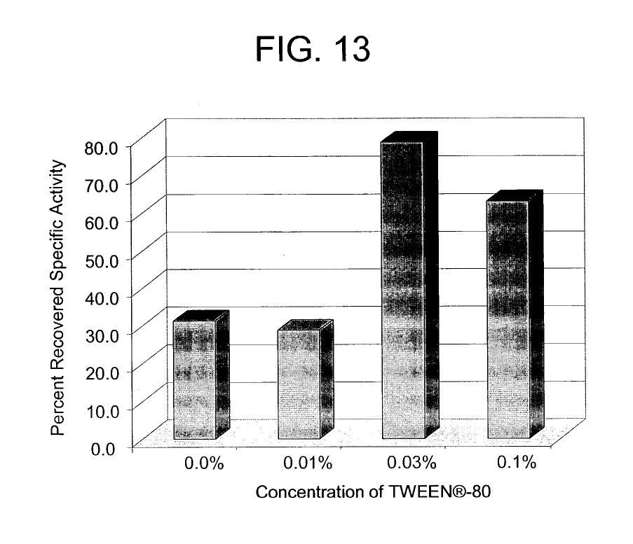

Active tPA was recovered following nebulization with TWEEN-80® surfactant (FIG. 13). Greater than 70% recovery of active tPA was achieved following nebulization with 0.03% TWEEN-80® surfactant (FIG. 13).

### Example 8

Nebulized tPA Inhibits ROS Production by Neutrophils

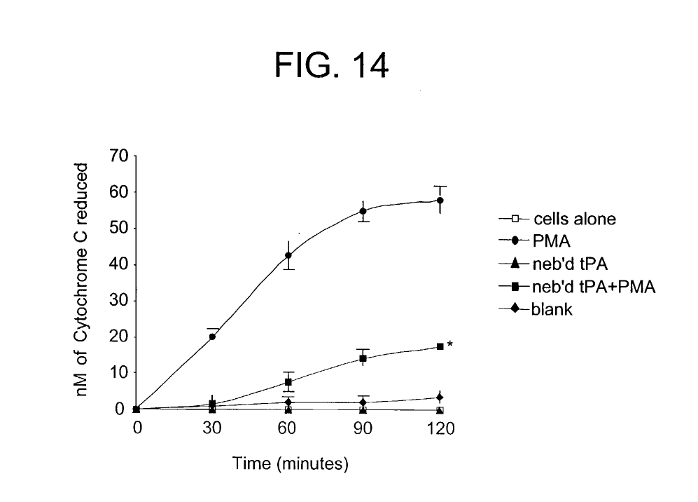

Nebulized tPA was prepared as described in Example 6. The ability of nebulized tPA to inhibit ROS production by neutrophils was determined by measuring cytochrome C reduction, as described in Example XX. Nebulized tPA inhibits PMA-induced ROS production (FIG. 14). In addition, incubation of neutrophils with nebulized tPA does not result in ROS production, and thus it is unlikely that the formulation for pulmonary delivery will result in significant activation of neutrophils.

Administration of Nebulized tPA in an Animal Model of Acute Lung Injury

This animal model is routinely used and works effectively to distribute instillate solution in the lungs. The administration protocol does not require invasive surgery. See e.g., Stringer et al. (1997b) Free Radic Biol Med 22:985-8, Gavett et al. (1995) J Exp Med 182:1527-36, and Hybertson et al. (1995) Free Radic Biol Med 18:537-42.

Treatment Groups. Animals (n=80) are exposed to either nebulized tPA or sham (vehicle) while contained in a nebulization chamber (Hybertson et al., 1998). Tissue plasminogen activator will be nebulized using a jet nebulizer (Side Stream nebulizer with Pulmo Aide compressor, available from DeVilbiss, Somerset, Pa.). The nebulizer has a 9-ml reservoir and an approximate rate of delivery of 0.44 ml/minute. Dose concentration studies are performed by adding 5 ml of reformulated tPA concentrations (e.g., 0, 10, 50, 100, 1000 μg/ml) to the nebulizer reservoir and nebulizing until the reservoir is empty (approximately 11 minutes). Thus, for example, a formulation containing 1000 μg/ml tPA will deliver a total of 5000 μg of tPA. These doses represent amounts that are anticipated to have no adverse effects (0 and 10 μg/ml) as well as doses that may be associated with significant toxicity (1000 μg/ml). See Stringer et al. (1997a) Inflammation 21:27-34 and Tebbe et al. (1989) Am J Cardiol 64:448-53.

Male Sprague-Dawley rats weighing 250-300 g (Sasco of Omaha, Nebr.) are allowed to acclimate for at least 7 days before study. Male rats are used for consistent animal size and to allow comparison to prior data. Gender-based differences in tPA response are not anticipated.

Reformulated tPA or vehicle is added to the nebulizer reservoir (5 ml). Four male Sprague-Dawley rats are placed in the chamber together and allowed to ambulate for 3-5 minutes prior to turning on the nebulizer. The nebulizer is allowed to run until the reservoir is empty (approximately 11 minutes).

Immediately following administration of nebulized vehicle or tPA, animals receive intratracheal instillation of IL-1 (50 ng/0.5 ml rhIL-α, available from R&D Systems of Minneapolis, Minn.) or vehicle (0.5 ml sterile saline), essentially as described by Stringer et al. (1997b) Free Radic Biol Med 22:985-8. Briefly, each animal is placed on an elevated platform in a glass jar with isoflurane-soaked 4 inch square gauze pads. After unconsciousness has been achieved (about 20-30 seconds), the animal is placed on its back on an inclined board, and gently held in place with a rubber band around its incisors. A 50-ml conical centrifuge tube containing isoflurane-soaked gauze is placed around the nose as needed to keep the animal unconscious. The tongue is gently pulled out and to the side to expose the trachea. Saline (0.5 ml) or IL-1 in saline (0.5 ml) is administered close to the epiglottis using a ball-tipped feeding needle. Two 3-ml puffs of air follow to promote distal delivery of the compounds in the lungs. The round tip of the needle is used to palpate the tracheal rings to assist proper location of the injection.

Measurements of Inflammatory Lung Injury. Following instillation, animals are allowed to regain consciousness and ambulate freely in a cage. Five hours after IL-1 or vehicle administration, inflammatory lung injury is assessed. Four of the animals in each treatment group are analyzed for myeloperoxidase (MPO) lung activity. The remaining four rats in each group are analyzed for lavage neutrophil counts and protein lung leak.

Myeloperoxidase activity is measured in lung tissue as an index of neutrophil concentration. At 5 hours after IL-1 or saline instillation, lungs are perfused blood-free with PBS and removed. Left lung samples are homogenized in 4.0 ml phosphate buffer (20 mM, pH 6.0). The homogenate is centrifuged at 18,000 rpm at 0-10° C. for 30 minutes. After discarding the supernatant, the pellet is resuspended in 4.0 ml phosphate buffer (50 mM, pH 6.0) with 5% hexadecyltrimethylammonium bromide and then frozen at −70° C. Samples are thawed, sonicated for 90 seconds, incubated at 60° C. for 2 hours (to inactivate tissue MPO inhibitors). The samples are then analyzed using o-dianisidine as substrate, essentially as described by Fulkerson et al. (1996) Arch Intern Med 156:29-38 and Snipes et al. (1989) Health Phys 57 Suppl 1:69-77; discussion 77-8.

At the time of insult, FITC-conjugated albumin is injected femorally. Five hours after IL-1 or saline instillation, animals are anesthetized by intraperitoneal administration of ketamine (90 mg/kg) and xylazine (7 mg/kg). Tracheotomy is performed, mechanical ventilation using a respirator is initiated, followed by laparotomy, thoracotomy, and then right ventricular injection of heparin (200 U, 0.2 ml).

Blood samples are then obtained from the right ventricle and the lungs are lavaged with saline. Saline (8.0 ml) is slowly injected and withdrawn three times. Recovered volume is measured, the bronchoalveolar lavage fluid is centrifuged and the supernatant is collected for further characterization. Protein concentration is measured by a bicinchroninic acid method (reagents available from Sigma of St. Louis, Mo.).

The leukocyte pellet is resuspended in 1.0 ml of supernatant. Total leukocytes are counted in a hemocytometer. A CYTOSPIN® apparatus (Shandon Southern Instruments Limited of Cheshire, England) is used to prepare samples, which are then stained with Wright-Giemsa to determine the percentage and total number of neutrophils.

The lungs are excised, homogenized, and centrifuged, and the supernatant is collected. Plasma is separated using a serofuge. Lavage, tissue homogenate supernatant, and plasma samples are assayed for FITC fluorescence (excitation 485 nm, emission 530 nm) using a microtiter plate fluorimeter. Lung leak index is examined as the ratio of background corrected fluorescence in 0.3 ml lavage fluid/0.3 ml plasma and in 0.3 ml tissue homogenate supernatant/0.3 ml plasma.

Data Analysis. A sample size of 80 animals allows for the detection of a 40% difference between groups (α=0.05, power [1β]=0.80). The mean (±SEM) for MPO activity, neutrophil count, and lung leak index for each treatment group and each dose of tPA is determined. Differences between control and treatment inflammatory measurements are compared using analysis of variance (ANOVA). ANOVA is also used to compare the dose response of tPA on the measured inflammatory markers. Ad hoc analysis is performed using a Student-Newman-Keuhls test to determine potential differences. In all cases, a p value of ≦0.05 is considered statistically significant. The statistical computer program STATVIEW® (Abacus Corp. of Berkeley, Calif.) is used to perform the analyses.

### Example 10

Pharmacokinetic Analysis of Nebulized tPA in vivo

Lung tissue samples are obtained as described in Example 9. The samples are analyzed for tPA concentration and compared to vehicle in order to verify that tPA is getting into the lungs. Lung tPA concentration (densitometry) in tPA-treated rats is anticipated to be at least 20,000 times greater than that of vehicle-treated rats. Dose is adjusted by increasing the concentration of formulated tPA and/or by increasing the duration of nebulization.

To monitor the absorption rate of tPA from the lungs into the systemic circulation, serial blood samples are collected from animals following the administration of tPA (e.g., 1, 3, or 6 mg/kg). Blood sampling is performed by tail-vein nicking. Each animal is anesthetized using inhaled isoflurane, as described in Example 9, just prior to the collection of each blood sample (300-500 μl) into a heparinized (100U/0.1 ml) EPPENDORF® tube (0.5 ml, available from Eppendorf AG Company of Hamburg, Germany).

Serial blood samples are collected from tPA-treated animals throughout the tPA nebulization procedure described in Example 9. A baseline (time 0) sample is obtained prior to placement of each animal in the nebulization chamber. Subsequent samples are collected at 30 and 60 minutes, and every hour thereafter. The last blood sample is collected via tail vein nicking just prior to the performance of inflammatory marker studies and euthanasia (approximately 5 hours). Upon collection, blood samples are immediately centrifuged at 150×g for 10 minutes. Plasma is transferred into a freezer tube and frozen at −80° C. until the time of assay. Blood samples are also collected from rats that receive both tPA and IL-1 to determine whether IL-1 administration alters tPA distribution and elimination.

For comparison, blood sampling is similarly performed following intraperitoneal administration of tPA, performed as described in Korninger & Collen (1981) Thromb Haemost 46:561-5.

tPA plasma concentrations are determined by ELISA using methods known in the art. Reference standards are prepared by reconstituting a 10-μg vial of tPA standard (Biopool International of Ventura, Calif.) with 1 ml of sterile water. Reconstituted tPA are added to tPA and PAI-1 depleted plasma to produce standards with final tPA concentrations of 50 ng/ml, 20 ng/ml, 10 ng/ml, 5 ng/ml, 2.5 ng/ml, and 1.25 ng/ml. A capture antibody (affinity purified sheep anti-tPA IgG, available from Enzyme Research of South Bend, Ind.) is diluted 1/100 in coating buffer (50 mM carbonate prepared using 1.59 g of Na₂CO₃ and 2.93 g of NaHCO₃ in 1 L H₂O, pH 9.6), and 100 μL is added to each well of a 96-well microplate. The plate is incubated overnight a 4° C. Following incubation and just prior to use, the contents of the plate are emptied and 150 μL of blocking buffer (2% BSA in PBS, pH 7.4) is added to each well. The plate is incubated for at least 60 minutes at 22° C. The plate is washed four times with PBS-TWEEN® (8 g NaCl, 2.9 g Na₂HPO₄.12H₂O, 0.2 g KH₂PO₄, 0.2 g KCl and 1 ml of TWEEN®-20 surfactant in 1 L H₂O, pH 7.4). Plasma samples are initially diluted 1:1000 with HBS-BSA-TWEEN®-20 (5.95 g HEPES, 1.46 g NaCl, 2.5 g BSA, and 0.25 ml TWEEN®-20 surfactant in 250 ml H₂O, pH 7.2) since they will most likely exceed the upper-limit of detection of the assay. See Tebbe et al. (1989) Am J Cardiol 64:448-53. To each well, 100 μL of sample or standard are added, and the plate is incubated at 22° C. for 90 minutes. The plate is washed four times with PBS-TWEEN®-20. The detecting antibody (peroxidase-conjugated sheep anti-tPA, available from Enzyme Research of South Bend, Ind.) is diluted 1/100 in HBS-BSA-TWEEN®-20 and 100 μL is added to each well. The plate is incubated at 22° C. for 90 minutes then washed four times with PBS-TWEEN®-20. OPD (O-phenylenediamine) substrate is prepared by dissolving 5 mg OPD in 12 ml of substrate buffer (citrate-phosphate buffer, pH 5.0) followed by the addition of 12 μL of H₂O₂ (3%). To each well, 100 μL of OPD substrate is added. Color is allowed to develop for 5 minutes, and the reaction is stopped with the addition of 2.5M H₂SO₄ (50 μl/well). The plate is read using a microtiter plate reader (ThermoMax, available from Molecular Devices Corporation of Sunnyvale, Calif.) at a wavelength of 490 nm. tPA concentration for each sample re determined from the standard curve plot (concentration vs. absorbance) which is calculated automatically by the plate reader's software.

Lung tPA concentration is also measured. Five hours following IL-1 or saline insufflation and euthanasia, the lower left lobe of the lung is removed from rats treated with nebulized tPA or vehicle as described in Example 9. Lung samples are also removed from animals receiving intravenous administration of tPA. Samples are flash frozen in liquid nitrogen and stored at −80° C. until the time of assay. Subsequently, samples are thawed on ice and homogenized with ice-cold homogenization buffer (20 nM HEPES/glycerol buffer, pH 7.5) containing protease inhibitors (2 mM EDTA, 2 mM EGTA, 5 μg/ml aprotonin, 10 μM leupeptin, 1 mM PMSF) and centrifuged at 15,000×g for 45 minutes. After the protein concentration of each supernatant has been determined, aliquots containing 100 μg protein are resolved by 7.5% acrylamide gel electrophoresis and transferred to nitrocellulose, essentially as described by Stringer et al. (1997b) Free Radic Biol Med 22:985-8. Membranes are blocked with 3% milk in TNS buffer (15 nM Tris, pH 7.4, 150 mM NaCl, 0.1% TWEEN®-20) overnight and then incubated with an antibody specific for tPA (1:50 dilution of affinity purified polyclonal sheep anti-tPA IgG antibody, available from Enzyme Research of South Bend, Ind.) for 60 minutes at 25° C. Blots are rinsed five times for 5 minutes in Western wash buffer (10×PBS, 10% TWEEN®-20 in water) and exposed to a secondary polyclonal antibody (1:10,000 dilution of rabbit anti-sheep horseradish peroxidase conjugate, available from Jackson ImmunoResearch of West Grove, Pa.] for 30 minutes at 25° C. Following five 5-minute rinses, immunoblots are visualized by application of chemiluminescence Western blotting reagents (available from NEN Life Science Products Inc. of Boston, Mass.) and exposure to autoradiographic film. Immunolabeled tPA is identified by comparison to a tPA standard (100 μg) and molecular weight markers included on the blot. Autoradiographic signal are quantified using video densitometry and data is analyzed using IMAGEQUANT® software (Molecular Dynamics of Sunnyvale, Calif.).

tPA elimination rate constant (k) is calculated following the intravenous administration of one of three tPA doses to 15 animals (n=5 animals/dose). The use of 5 animals per dose allows detection of a 50% difference in measured peak plasma tPA concentration between doses using an □=0.05 and power [1-ε]=0.80.

Mean (±SEM) plasma tPA concentration versus time curves are plotted for each nebulized and intravenous dose of tPA. The rate of tPA absorption from the lungs is approximated using the Loo-Riegelman method, which allows determination of absorption rate independent of the dose of tPA delivered to the lungs. See Gibaldi & Perrier (1982) Pharmacokinetics, Marcel Dekker, New York. This principle assumes a multi-compartment model (tPA has been shown to follow two or three compartment kinetics) and first-order (linear) absorption from the lungs (Godfrey et al., 1998; Tebbe et al., 1989). plasma tPA concentration-time data following pulmonary or intravenous administration of tPA is then used to calculate the fraction of drug absorbed from the lung.

### REFERENCES

The references listed below as well as all references cited in the specification are each incorporated herein by reference in their entirety to thereby supplement, explain, provide a background for or teach methodology, techniques and/or compositions employed herein.

American Lung Association statistics; adult (acute) respiratory distress syndrome (ARDS). http://www.lungusa.org.

Abraham E, Matthay M A, Dinarello C A, Vincent J L, Cohen J, Opal S M, Glauser M, Parsons P, Fisher C J, Jr. & Repine J E (2000). Consensus conference definitions for sepsis, septic shock, acute lung injury, and acute respiratory distress syndrome: time for a reevaluation. Crit Care Med 28:232-235.

Ando Y, lnoue M, Araki S & Morino Y (1991). Inhibition of carrageenin-induced paw edema by a superoxide dismutase derivative that circulates bound to albumin. Biochim Biophys Acta 1073:374-379.

Appella E, Robinson E A, Ullrich S J, Stoppelli M P, Corti A, Cassani G & Blasi F (1987). The receptor-binding sequence of urokinase. A biological function for the growth-factor module of proteases. J Biol Chem 262:4437-4440.

Archer S L, Nelson D P & Weir E K (1989). Detection of activated O₂ species in vitro and in rat lungs by chemiluminescence. J Appl Physiol 67:1912-1921.

Artigas A, Bernard G R, Carlet J, Dreyfuss D, Gattinoni L, Hudson L, Lamy M, Marini J J, Matthay M A, Pinsky M R, et al. (1998). The American-European Consensus Conference on ARDS, part 2: Ventilatory, pharmacologic, supportive therapy, study design strategies, and issues related to recovery and remodeling. Acute respiratory distress syndrome. Am J Respir Crit Care Med 157:1332-1347.

Ausubel (ed.) (1995) Short Protocols in Molecular Biology, 3rd ed. Wiley, New York.

Babior B M, Kipnes R S & Curnutte J T (1973). Biological defense mechanisms. The production by leukocytes of superoxide, a potential bactericidal agent. J Clin Invest 52:741-744.

Bell D, Jackson M, Nicoll J J, Millar A, Dawes J & Muir A L (1990). Inflammatory response, neutrophil activation, and free radical production after acute myocardial infarction: effect of thrombolytic treatment. Br Heart J 63:82-87.

Berkow et al. (1997) The Merck Manual of Medical Information, Home ed. Merck Research Laboratories, Whitehouse Station, N.J.

Bertozzi P, Astedt B, Zenzius L, Lynch K, LeMaire F, Zapol W & Chapman H A, Jr. (1990). Depressed bronchoalveolar urokinase activity in patients with adult respiratory distress syndrome. N Engl J Med 322:890-897.

Cheng Y S, Holmes T D, Gao J, Guilmette R A, Li S, Surakitbanharn Y & Rowlings C (2001 b). Characterization of nasal spray pumps and deposition pattern in a replica of the human nasal airway. J Aerosol Med 14:267-280.

Cooper J A, Lo S K & Malik A B (1988). Fibrin is a determinant of neutrophil sequestration in the lung. Circ Res 63:735-741.

Dakik H A & Nasrallah A (2001). Repeated doses of tissue plasminogen activator for failed thrombolysis: case report and review of the literature. Heart Dis 3:362-364.

Davidson T A, Rubenfeld G D, Caldwell E S, Hudson L D & Steinberg K P (1999). The effect of acute respiratory distress syndrome on long-term survival. Am J Respir Crit Care Med 160:1838-1842.

Deby C & Goutier R (1990). New perspectives on the biochemistry of superoxide anion and the efficiency of superoxide dismutases. Biochem Pharmacol 39:399-405.

Demling R H (1995). The modern version of adult respiratory distress syndrome. Annu Rev Med 46:193-202.

Desai S R (2002). Acute respiratory distress syndrome: imaging of the injured lung. Clin Radiol 57:8-17.

Doring G (1994). The role of neutrophil elastase in chronic inflammation. Am J RespirCrit Care Med 150:S114-117.

Dunn B M, Scarborough P E, Davenport R & Swietnicki W (1994). Analysis of proteinase specificity by studies of peptide substrates. The use of UV and fluorescence spectroscopy to quantitate rates of enzymatic cleavage. Methods Mol Biol 36:225-243.

Duke et al. (1995). In Current Protocols in Immunology, Coligan et al., ed. (New York, John Wiley & Sons), pp. 3.17.11-13.17.33.

Ebadi (1998) CRC Desk Reference of Clinical Pharmacology. CRC Press, Boca Raton, Fla.

Fantone J C & Kinnes D A (1983). Prostaglandin E1 and prostaglandin 12 modulation of superoxide production by human neutrophils. Biochem Biophys Res Commun 113:506-512.

Ferrante A & Thong Y H (1980). Optimal conditions for simultaneous purification of mononuclear and polymorphonuclear leucocytes from human blood by the Hypaque-Ficoll method. J Immunol Methods 36:109-117.

Fulkerson W J, Maclntyre N, Stamler J & Crapo J D (1996). Pathogenesis and treatment of the adult respiratory distress syndrome. Arch Intern Med 156:29-38.

Fults K, Cyr T D & Hickey A J (1991). The influence of sampling chamber dimensions on aerosol particle size measurement by cascade impactor and twin impinger. J Pharm Pharmacol 43:726-728.

Gavett S H, O'Hearn D J, Li X, Huang S K, Finkelman F D & Wills-Karp M (1995). Interleukin 12 inhibits antigen-induced airway hyperresponsiveness, inflammation, and Th2 cytokine expression in mice. J Exp Med 182:1527-1536.

Gibaldi M & Perrier D (1982). Pharmacokinetics (New York, Marcel Dekker).

Glover & Hames (1995) DNA Cloning: A Practical Approach, 2nd ed. IRL Press at Oxford University Press, Oxford/New York.

Godfrey K R, Tanswell P, Bates R A, Chappell M J & Madden F N (1998). Nonlinear pharmacokinetics of tissue-type plasminogen activator in three animal species: a comparison of mathematical models. Biopharm Drug Dispos 19:131-140.

Goldstein I M, Kaplan H B, Edelson H S & Weissmann G (1982). Ceruloplasmin: an acute phase reactant that scavenges oxygen-derived free radicals. Ann N Y Acad Sci 389:368-379.

Goodman et al. (1996) Goodman & Gilman's the Pharmacological Basis of Therapeutics, 9th ed. McGraw-Hill Health Professions Division, New York.

Grieg A (1998). Thrombolysis of a neonatal brachial artery thrombosis with tissue plasminogen activator. J Perinatol 18:460-462.

Gunther A, Ruppert C, Schmidt R, Markart P, Grimminger F, Walmrath D & Seeger W (2001). Surfactant alteration and replacement in acute respiratory distress syndrome. Respir Res 2:353-364.

Gurewich V, Pannell R, Broeze R J & Mao J (1988). Characterization of the intrinsic fibrinolytic properties of pro-urokinase through a study of plasmin-resistant mutant forms produced by site-specific mutagenesis of lysine (158). J Clin Invest 82:1956-1962.

Hajjar K A, Hamel N M, Harpel P C & Nachman R L (1987). Binding of tissue plasminogen activator to cultured human endothelial cells. J Clin Invest 80:1712-1719.

Hansen P R (1995). Role of neutrophils in myocardial ischemia and reperfusion. Circulation 91:1872-1885.

Hart D A & Fritzler M J (1989). Regulation of plasminogen activators and their inhibitors in rheumatic diseases: new understanding and the potential for new directions. J Rheumatol 16:1184-1191.

Hybertson B (1994). Anal Lett 127:3081-3093.

Hybertson B M, Kitlowski R P, Jepson E K & Repine J E (1998). Supercritical fluid-aerosolized vitamin E pretreatment decreases leak in isolated oxidant-perfused rat lungs. J Appl Physiol 84:263-268.

Hybertson B M, Leff J A, Beehler C J, Barry P C & Repine J E (1995). Effect of vitamin E deficiency and supercritical fluid aerosolized vitamin E supplementation on interleukin-1-induced oxidative lung injury in rats. Free Radic Biol Med 18:537-542.

Idell S, James K K, Levin E G, Schwartz B S, Manchanda N, Maunder R J, Martin T R, McLarty J & Fair D S (1989). Local abnormalities in coagulation and fibrinolytic pathways predispose to alveolar fibrin deposition in the adult respiratory distress syndrome. J Clin Invest 84:695-705.

Idell S, Koenig K B, Fair D S, Martin T R, McLarty J & Maunder R J (1991). Serial abnormalities of fibrin turnover in evolving adult respiratory distress syndrome. Am J Physiol 261:L240-248.

Janoff A (1985). Elastase in tissue injury. Annu Rev Med 36:207-216.

Katzung (2001) Basic & Clinical Pharmacology, 8th ed. Lange Medical Books/McGraw-Hill Medical Pub. Division, New York.

Kehl H G, Kececioglu D, Vielhaber H, Kotthoff S, Weyand M, Jorch G, Vogt J & Nowak-Gottl U (1996). Left atrial thrombus in a 10-month-old boy—successful thrombolysis with recombinant tissue-type plasminogen activator after open-heart surgery: review of the literature. Intensive Care Med 22:968-971.

Kim Y S, Cape S P, Chi E, Raffen R, Wilkins-Stevens P, Stevens F J, Manning M C, Randolph T W, Solomon A & Carpenter J F (2001). Counteracting effects of renal solutes on amyloid fibril formation by immunoglobulin light chains. J Biol Chem 276:1626-1633.

Kirk C J & Mule J J (2000). Gene-modified dendritic cells for use in tumor vaccines. Hum Gene Ther 11:797-806.

Koh Y, Hybertson B M, Jepson E K, Cho O J & Repine J E (1995). Cytokine-induced neutrophil chemoattractant is necessary for interleukin-1-induced lung leak in rats. J Appl Physiol 79:472-478.

Korninger C & Collen D (1981). Studies on the specific fibrinolytic effect of human extrinsic (tissue-type) plasminogen activator in human blood and in various animal species in vitro. Thromb Haemost 46:561-565.

Krawisz J E, Sharon P & Stenson W F (1984). Quantitative assay for acute intestinal inflammation based on myeloperoxidase activity. Assessment of inflammation in rat and hamster models. Gastroenterology 87:1344-1350.

Leff J A, Baer J W, Bodman M E, Kirkman J M, Shanley P F, Patton L M, Beehler C J, McCord J M & Repine J E (1994). Interleukin-1-induced lung neutrophil accumulation and oxygen metabolite-mediated lung leak in rats. Am J Physiol 266:L2-8.

Leff J A, Wilke C P, Hybertson B M, Shanley P F, Beehler C J & Repine J E (1993). Postinsult treatment with N-acetyl-L-cysteine decreases IL-1-induced neutrophil influx and lung leak in rats. Am J Physiol 265:L501-506.

Lijnen H R, Uytterhoeven M & Collen D (1984). Inhibition of trypsin-like serine proteinases by tripeptide arginyl and lysyl chloromethylketones. Thromb Res 34:431-437.

Mackensen A, Lindemann A & Mertelsmann R (1997). Immunostimulatory cytokines in somatic cells and gene therapy of cancer. Cytokine Growth Factor Rev 8:119-128.

MacIntyre N A view to the future of treatments for acute lung injury. 96th International Conference of the American Thoracic Society.

Magdolen V, Rettenberger P, Koppitz M, Goretzki L, Kessler H, Weidle U H, Konig B, Graeff H, Schmitt M & Wilhelm O (1996). Systematic mutational analysis of the receptor-binding region of the human urokinase-type plasminogen activator. Eur J Biochem 237:743-751.

Marshall B C, Sageser D S, Rao N V, Emi M & Hoidal J R (1990). Alveolar epithelial cell plasminogen activator. Characterization and regulation. J Biol Chem 265:8198-8204.

McLean J A, Minnich M G, Montaser A, Su J & Lai W (2000). Optical patternation: a technique for three-dimensional aerosol diagnostics. Anal Chem 72:4796-4804.

McLoud T C (2002). Imaging techniques for diagnosis and staging of lung cancer. Clin Chest Med 23:123-136.

McWilliams A, MacAulay C, Gazdar A F & Lam S (2002). Innovative molecular and imaging approaches for the detection of lung cancer and its precursor lesions. Oncogene 21:6949-6959.

Munkvad S (1993). Fibrinolysis in patients with acute ischaemic heart disease. With particular reference to systemic effects of tissue-type plasminogen activator treatment on fibrinolysis, coagulation and complement pathways. Dan Med Bull 40:383-408.

Ny T, Elgh F & Lund B (1984). The structure of the human tissue-type plasminogen activator gene: correlation of intron and exon structures to functional and structural domains. Proc Natl Acad Sci USA 81:5355-5359.

Pavey S J, Hawson G A & Marsh N A (1999). Alterations to the fibrinolytic enzyme system in patients with non-small cell lung carcinoma. Blood Coagul Fibrinolysis 10:261-267.

Pennica D, Holmes W E, Kohr W J, Harkins R N, Vehar G A, Ward C A, Bennett W F, Yelverton E, Seeburg P H, Heyneker H L, et al. (1983). Cloning and expression of human tissue-type plasminogen activator cDNA in E. coli. Nature 301:214-221.

Remington et al. (1975) Remington's Pharmaceutical Sciences, 15th ed. Mack Pub. Co., Easton, Pa.

Riccio A, Grimaldi G, Verde P, Sebastio G, Boast S & Blasi F (1985). The human urokinase-plasminogen activator gene and its promoter. Nucleic Acids Res 13:2759-2771.

Riesenberg K, Schlaeffer F, Katz A & Levy R (1995). Inhibition of superoxide production in human neutrophils by combinations of heparin and thrombolytic agents. Br Heart J 73:14-19.

Ruiz M A, Marugan I, Estelles A, Navarro I, Espana F, Alberola V, San Juan L, Aznar J & Garcia-Conde J (1989). The influence of chemotherapy on plasma coagulation and fibrinolytic systems in lung cancer patients. Cancer 63:643-648.

Sambrook et al. (eds.) (1989) Molecular Cloning: A Laboratory Manual. Cold Spring Harbor Laboratory Press, Cold Spring Harbor, N.Y.

Schermuly R T, Gunther A, Weissmann N, Ghofrani H A, Seeger W, Grimminger F & Walmrath D (2000). Differential impact of ultrasonically nebulized versus tracheal-instilled surfactant on ventilation-perfusion (VA/Q) mismatch in a model of acute lung injury. Am J Respir Crit Care Med 161:152-159.

Siegel M. Novel therapies for the acute respiratory distress syndrome. Silhavy et al. (1984) Experiments with Gene Fusions. Cold Spring Harbor Laboratory Press, Cold Spring Harbor, N.Y.

Slutsky A NHLBI ARDSnet: Report of ongoing and proposed clinical trials. 96th International Conference of the American Thoracic Society.

Snipes M B, McClellan R O, Mauderly J L & Wolff R K (1989). Retention patterns for inhaled particles in the lung: comparisons between laboratory animals and humans for chronic exposures. Health Phys 57 Suppl 1:69-77; discussion 77-68.

Speight et al. (1997) Avery's Drug Treatment: A Guide to the Properties, Choice, Therapeutic Use and Economic Value of Drugs in Disease Management, 4th ed. Adis International, Auckland/Philadelphia, Pa.

Stoppelli M P, Corti A, Soffientini A, Cassani G, Blasi F & Assoian R K (1985). Differentiation-enhanced binding of the amino-terminal fragment of human urokinase plasminogen activator to a specific receptor on U937 monocytes. Proc Natl Acad Sci USA 82:4939-4943.

Stringer K A (2000). Tissue plasminogen activator inhibits reactive oxygen species production by macrophages. Pharmacotherapy 20:375-379.

Stringer K A, Bose S K & McCord J M (1997a). Antiinflammatory activity of tissue plasminogen activator in the carrageenan rat footpad model. Free Radic Biol Med 22:985-988.

Stringer K A, Lindenfeld J, Repine A J, Cohen Z & Repine J E (1997b). Tissue plasminogen activator (tPA) inhibits human neutrophil superoxide anion production in vitro. Inflammation 21:27-34.

Tebbe U, Tanswell P, Seifried E, Feuerer W, Scholz K H & Herrmann K S (1989). Single-bolus injection of recombinant tissue-type plasminogen activator in acute myocardial infarction. Am J Cardiol 64:448-453.

Towbin H, Staehelin T & Gordon J (1979). Electrophoretic transfer of proteins from polyacrylamide gels to nitrocellulose sheets: procedure and some applications. Proc Natl Acad Sci USA 76:4350-4354.

Ts'ao CH, Ward W F & Port C D (1983). Radiation injury in rat lung. III. Plasminogen activator and fibrinolytic inhibitor activities. Radiat Res 96:301-308.

U.S. Patent Application Serial No. 60/036,566

U.S. patent application Ser. No. 09/355,522

U.S. Pat. No. 4,326,033

U.S. Pat. No. 4,370,417

U.S. Pat. No. 4,766,075

U.S. Pat. No. 4,963,357

U.S. Pat. No. 4,976,959

U.S. Pat. No. 5,057,494

U.S. Pat. No. 5,075,222-611

U.S. Pat. No. 5,094,953

U.S. Pat. No. 5,106,741

U.S. Pat. No. 5,108,901

U.S. Pat. No. 5,112,755

U.S. Pat. No. 5,149,533

U.S. Pat. No. 5,156,969

U.S. Pat. No. 5,175,105

U.S. Pat. No. 5,219,569

U.S. Pat. No. 5,232,847

U.S. Pat. No. 5,240,845

U.S. Pat. No. 5,242,688

U.S. Pat. No. 5,246,850

U.S. Pat. No. 5,270,198

U.S. Pat. No. 5,275,946

U.S. Pat. No. 5,277,175

U.S. Pat. No. 5,284,133

U.S. Pat. No. 5,299,566

U.S. Pat. No. 5,355,872

U.S. Pat. No. 5,359,032

U.S. Pat. No. 5,420,110

U.S. Pat. No. 5,453,490

U.S. Pat. No. 5,455,229

U.S. Pat. No. 5,455,330

U.S. Pat. No. 5,472,692

U.S. Pat. No. 5,486,471

U.S. Pat. No. 5,510,333

U.S. Pat. No. 5,519,120

U.S. Pat. No. 5,521,185

U.S. Pat. No. 5,525,623

U.S. Pat. No. 5,541,288

U.S. Pat. No. 5,550,213

U.S. Pat. No. 5,556,621

U.S. Pat. No. 5,571,708

U.S. Pat. No. 5,660,166

U.S. Pat. No. 5,756,512

U.S. Pat. No. 5,795,871

U.S. Pat. No. 5,797,389

U.S. Pat. No. 5,823,179

U.S. Pat. No. 6,016,974

U.S. Pat. No. 6,041,776

U.S. Pat. No. 6,044,841

U.S. Pat. No. 6,241,159

U.S. Pat. No. 6,242,204

U.S. Pat. No. 6,354,516

U.S. Pat. No. 6,357,671

U.S. Published Patent Application No. 20020020409

U.S. Published Patent Application No. 20020020412

Vecellio None L, Grimbert D, Becquemin M H, Boissinot E, Le Pape A, Lemarie E & Diot P (2001). Validation of laser diffraction method as a substitute for cascade impaction in the European Project for a Nebulizer Standard. J Aerosol Med 14:107-114.

Vinegar R, Schreiber W & Hugo R (1969). Biphasic development of carrageenin edema in rats. J Pharmacol Exp Ther 166:96-103.

Vinegar R, Truax J F & Selph J L (1976). Quantitative studies of the pathway to acute carrageenan inflammation. Fed Proc 35:2447-2456.

Vinegar R, Truax J F, Selph J L, Johnston P R, Venable A L & McKenzie K K (1987). Pathway to carrageenan-induced inflammation in the hind limb of the rat. Fed Proc 46:118-126.

Walther W & Stein U (1999). Therapeutic genes for cancer gene therapy. Mol Biotechnol 13:21-28.

Waud W R, Brady F O, Wiley R D & Rajagopalan K V (1975). A new purification procedure for bovine milk xanthine oxidase: effect of proteolysis on the subunit structure. Arch Biochem Biophys 169:695-701.

Weissmann G, Serhan C, Korchak H M & Smolen J E (1982). Neutrophils: release of mediators of inflammation with special reference to rheumatoid arthritis. Ann N Y Acad Sci 389:11-24.

Wiernikowski J T, Crowther M, Clase C M, Ingram A, Andrew M & Chan A K (2000). Stability and sterility of recombinant tissue plasminogen activator at −30 degrees C. Lancet 355:2221-2222.

Williams R O, 3rd, Repka M A & Barron M K (1999). Application of co-grinding to formulate a model pMDI suspension. Eur J Pharm Biopharm 48:131-140.

Winter C A & Flataker L (1965). Reaction thresholds to pressure in edematous hindpaws of rats and responses to analgesic drugs. J Pharmacol Exp Ther 150:165-171.

Zivkovic I, Thompson D M, Bertolino M, Uzunov D, DiBella M, Costa E & Guidotti A (1995). 7-Chloro-3-methyl-3-4-dihydro-2H-1,2,4 benzothiadiazine S,S-dioxide (IDRA 21): a benzothiadiazine derivative that enhances cognition by attenuating DL-alpha-amino-2,3-dihydro-5-methyl-3-oxo-4-isoxazolepropanoic acid (AMPA) receptor desensitization. J Pharmacol Exp Ther 272:300-309.

While the present invention has been described in connection with what is presently considered to be practical and preferred embodiments, it is understood that the present invention is not to be limited or restricted to the disclosed embodiments but, on the contrary, is intended to cover various modifications and equivalent arrangements included within the scope of the appended claims.

Thus, it is to be understood that variations in the described invention will be obvious to those skilled in the art without departing from the novel and non-obvious aspects of the present invention, and such variations are intended to come within the scope of the claims below.

What is claimed is:

1. A composition suitable for aerosolization comprising a biologically active protein and a non-physiological surfactant, wherein the non-physiological surfactant is included in an amount greater than the CMC of the surfactant.

2. The composition of claim 1, wherein the protein comprises a therapeutic protein or a diagnostic protein.

3. The composition of claim 1, wherein the protein comprises a plasminogen activator.

4. The composition of claim 3, wherein the plasminogen activator comprises tissue-type plasminogen activator or urokinase plasminogen activator.

5. The composition of claim 4, wherein the plasminogen activator comprises tissue-type plasminogen activator.

6. The composition of claim 1, wherein the protein comprises a human protein.

7. The composition of claim 1, wherein the surfactant comprises a non-ionic surfactant or an ionic surfactant.

8. The composition of claim 7, wherein the surfactant comprises a non-ionic surfactant.

9. The composition of claim 8, wherein the non-ionic surfactant comprises a block copolymer surfactant.

10. The composition of claim 9, wherein the block copolymer surfactant comprises a block copolymer of propylene oxide and ethylene oxide.

11. The composition of claim 9, wherein the block copolymer comprises a PLURONIC® surfactant.

12. The composition of claim 11, wherein the PLURONIC® surfactant comprises PLURONIC®-F68 surfactant.

13. The composition of claim 8, wherein the non-ionic surfactant comprises a polysorbate.

14. The composition of claim 13, wherein the polysorbate comprises polysorbate 80.

15. The composition of claim 13, wherein the polysorbate comprises a TWEEN® surfactant.

16. The composition of claim 15, wherein the TWEEN® surfactant comprises TWEEN®-80 surfactant.

17. The composition of claim 1, wherein the surfactant further comprises an amount from 0.01% (w/w) to 0.5% (w/w).

18. The composition of claim 17, wherein the surfactant further comprises an amount from 0.03% (w/w) to 0.5% (w/w).

19. The composition of claim 18, wherein the surfactant further comprises an amount from 0.05% (w/w) to 0.5% (w/w).

20. The composition of claim 18, wherein the surfactant further comprises an amount from 0.1% (w/w) to 0.5% (w/w).

21. The composition of claim 1, wherein the surfactant further comprises an amount about 0.1% (w/w).

22. The composition of claim 1, further comprising fibrinolytic activity.

23. The composition of claim 1, further comprising anti-inflammatory activity.

24. The composition of claim 23, wherein the anti-inflammatory activity comprises inhibition of reactive oxygen species production.

25. The composition of claim 23, wherein the anti-inflammatory activity comprises inhibition of lung leak.

27. The composition of claim 23, further comprising fibrinolytic activity.

28. The composition of claim 1, further comprising a detectable label.

29. A method for preparing an aerosol composition, the method comprising:
(a) preparing a composition comprising a biologically active protein and a non-physiological surfactant, wherein the non-physiological surfactant is included in an amount greater than the CMC of the surfactant; and
(c) aerosolizing the composition of (a).

30. The method of claim 29, wherein the protein comprises a therapeutic protein or a diagnostic protein.

31. The method of claim 29, wherein the protein comprises a plasminogen activator.

32. The method of claim 31, wherein the plasminogen activator comprises tissue-type plasminogen activator or urokinase plasminogen activator.

33. The method of claim 32, wherein the plasminogen activator comprises tissue-type plasminogen activator.

34. The method of claim 29, wherein the surfactant comprises a non-ionic surfactant or an ionic surfactant.

35. The method of claim 34, wherein the surfactant comprises a non-ionic surfactant.

36. The method of claim 35, wherein the non-ionic surfactant comprises a block copolymer surfactant.

37. The method of claim 36, wherein the block copolymer surfactant comprises a block copolymer of propylene oxide and ethylene oxide.

38. The method of claim 36, wherein the block copolymer comprises a PLURONIC®) surfactant.

39. The method of claim 38, wherein the PLURONIC® surfactant comprises PLURONIC®-F68 surfactant.

40. The method of claim 35, wherein the non-ionic surfactant comprises a polysorbate.

41. The method of claim 40, wherein the polysorbate comprises polysorbate 80.

42. The method of claim 40, wherein the polysorbate comprises a TWEEN® surfactant.

43. The method of claim 42, wherein the TWEEN® surfactant comprises TWEEN®-80 surfactant.

44. The method of claim 29, wherein the surfactant further comprises an amount from 0.01% (w/w) to 0.5% (w/w).

45. The method of claim 44, wherein the surfactant further comprises an amount from 0.03% (w/w) to 0.5% (w/w).

46. The method of claim 45, wherein the surfactant further comprises an amount from 0.05% (w/w) to 0.5% (w/w).

47. The method of claim 46, wherein the surfactant further comprises an amount from 0.1% (w/w) to 0.5% (w/w).

48. The method of claim 29, wherein the surfactant further comprises an amount about 0.1% (w/w).

49. The method of claim 29, wherein the composition further comprises a detectable label.

50. The method of claim 29, wherein the aerosolizing comprises performing jet nebulization or ultrasonic nebulization.

51. The method of claim 29, wherein the aerosolizing comprises using a metered dose inhaler.

52. The method of claim 29, wherein the aerosolizing comprises passaging the composition through a nozzle.

53. An aerosol composition produced by the method of claim 29.

54. The composition of claim 53, further comprising fibrinolytic activity.

55. The composition of claim 53, further comprising anti-inflammatory activity.

56. The composition of claim 55, wherein the anti-inflammatory activity comprises inhibition of reactive oxygen species production.

57. The composition of claim 55, wherein the anti-inflammatory activity comprises inhibition of lung leak.

58. The composition of claim 55, further comprising fibrinolytic activity.

59. A method for pulmonary delivery of a biologically active protein to a subject, the method comprising administering an effective amount of an aerosolized surfactant composition, wherein the composition comprises a biologically active protein and a non-physiological surfactant, and wherein the non-physiological surfactant is included in an amount greater than the CMC of the surfactant.

60. The method of claim 59, wherein the subject is a mammal.

61. The method of claim 60, wherein the mammal is a human.

62. The method of claim 59, wherein the subject comprises a lung disease or disorder.

63. The method of claim 62, wherein the lung disease or disorder comprises an inflammatory disease or disorder.

64. The method of claim 63 wherein said inflammatory lung disease is selected from the group consisting of acute lung injury, acute respiratory distress syndrome, asthma, bronchitis, and cystic fibrosis.

65. The method of 64 where the inflammatory lung disease is acute respiratory distress syndrome (ARDS).

66. The method of claim 62, wherein the lung disease or disorder comprises an embolism.

67. The method of claim 62, wherein the lung disease or disorder comprises cancer.

68. The method of claim 59, wherein the protein comprises a therapeutic protein or a diagnostic protein.

69. The method of claim 59, wherein the protein comprises a plasminogen activator.

70. The method of claim 59, wherein the plasminogen activator comprises tissue-type plasminogen activator or urokinase plasminogen activator.

71. The method of claim 70, wherein the plasminogen activator comprises tissue-type plasminogen activator.

72. The method of claim 59, wherein the surfactant comprises a non-ionic surfactant or an ionic surfactant.

73. The method of claim 72, wherein the surfactant comprises a non-ionic surfactant.

74. The method of claim 73, wherein the non-ionic surfactant comprises a block copolymer surfactant.

75. The method of claim 74, wherein the block copolymer surfactant comprises a block copolymer of propylene oxide and ethylene oxide.

76. The method of claim 74, wherein the block copolymer comprises a PLURONIC® surfactant.

77. The method of claim 76, wherein the PLURONIC® surfactant comprises PLURONIC®-F68 surfactant.

78. The method of claim 73, wherein the non-ionic surfactant comprises a polysorbate.

79. The method of claim 78, wherein the polysorbate comprises polysorbate 80.

80. The method of claim 78, wherein the polysorbate comprises a TWEEN® surfactant.

81. The method of claim 80, wherein the TWEEN® surfactant comprises TWEEN®-80 surfactant.

82. The method of claim 59, wherein the surfactant further comprises an amount from 0.01% (w/w) to 0.5% (w/w).

83. The method of claim 82, wherein the surfactant further comprises an amount from 0.03% (w/w) to 0.5% (w/w).

84. The method of claim 83, wherein the surfactant further comprises an amount from 0.05% (w/w) to 0.5% (w/w).

85. The method of claim 84, wherein the surfactant further comprises an amount from 0.1% (w/w) to 0.5% (w/w).

86. The method of claim 59, wherein the surfactant further comprises an amount about 0.1% (w/w).

87. The method of claim 59, further comprising treating a lung disease or disorder.

88. The method of claim 87, wherein the lung disease or disorder comprises an inflammatory disease or disorder, and whereby inflammation or inflammation-dependent lung damage in the subject is reduced.

89. The method of claim 88 wherein said inflammatory lung disease is selected from the group consisting of acute lung injury, acute respiratory distress syndrome, asthma, bronchitis, and cystic fibrosis.

90. The method of 89 where the inflammatory lung disease is acute respiratory distress syndrome (ARDS).

91. The method of claim 87, wherein the lung disease or disorder comprises an embolism, and whereby the embolism is reduced.

92. The method of claim 87, wherein the lung disease or disorder comprises cancer, and whereby cancer growth is reduced.

93. The method of claim 59, wherein the composition further comprises a detectable label.

94. The method of claim 93, further comprising detecting the detectable label.

95. A method for inhibiting pulmonary inflammation in a subject comprising administering to a subject an effective amount of an aerosolized surfactant composition, wherein the composition comprises a biologically active tissue-type plasminogen activator and a non-physiological surfactant, wherein the non-physiological surfactant is included in an amount greater than the CMC of the surfactant, and whereby pulmonary inflammation is reduced in the subject.

96. The method of claim 95, wherein the subject is a mammal.

97. The method of claim 96, wherein the mammal is a human.

98. The method of claim 95 wherein the subject comprises and inflammatory lung disease selected from the group consisting of acute lung injury, acute respiratory distress syndrome, asthma, bronchitis, and cystic fibrosis.

99. The method of 98 where the inflammatory lung disease is acute respiratory distress syndrome (ARDS).

100. The method of claim 95, wherein the surfactant comprises a non-ionic surfactant or an ionic surfactant.

101. The method of claim 100, wherein the surfactant comprises a non-ionic surfactant.

102. The method of claim 101, wherein the non-ionic surfactant comprises a block copolymer surfactant.

103. The method of claim 102, wherein the block copolymer surfactant comprises a block copolymer of propylene oxide and ethylene oxide.

104. The method of claim 103, wherein the block copolymer comprises a PLURONIC® surfactant.

105. The method of claim 103, wherein the PLURONIC® surfactant comprises PLURONIC®-F68 surfactant.

106. The method of claim 100, wherein the non-ionic surfactant comprises a polysorbate.

107. The method of claim 106, wherein the polysorbate comprises polysorbate 80.

108. The method of claim 106, wherein the polysorbate comprises a TWEEN® surfactant.

109. The method of claim 108, wherein the TWEEN® surfactant comprises TWEEN®-80 surfactant.

110. The method of claim 95, wherein the surfactant further comprises an amount from 0.01% (w/w) to 0.5% (w/w).

111. The method of claim 110, wherein the surfactant further comprises an amount from 0.03% (w/w) to 0.5% (w/w).

112. The method of claim 111, wherein the surfactant further comprises an amount from 0.05% (w/w) to 0.5% (w/w).

113. The method of claim 112, wherein the surfactant further comprises an amount from 0.1% (w/w) to 0.5% (w/w).

114. The method of claim 95, wherein the surfactant further comprises an amount about 0.1% (w/w).
